(19) United States
(12) Patent Application Publication
(10) Pub. No.: US 2025/0130232 A1
(43) Pub. Date: Apr. 24, 2025

(54) METHODS OF LOADING A STREPTAVIDIN COLUMN WITH BIOTINYLATED AFFINITY AGENTS

(71) Applicant: Waters Technologies Corporation, Milford, MA (US)

(72) Inventors: Kevin Wyndham, Worcester, MA (US); Beatrice Muriithi, Attleboro, MA (US); Martin Gilar, Franklin, MA (US); Yeliz Tunc Sarisozen, Westford, MA (US)

(73) Assignee: Waters Technologies Corporation, Milford, MA (US)

(21) Appl. No.: 19/005,819

(22) Filed: Dec. 30, 2024

Related U.S. Application Data
(63) Continuation of application No. 18/647,863, filed on Apr. 26, 2024.
(60) Provisional application No. 63/555,315, filed on Feb. 19, 2024.
(60) Provisional application No. 63/499,094, filed on Apr. 28, 2023.

(51) Int. Cl. G01N 33/569; G01N 33/53; G01N 33/543; G01N 33/74

(52) U.S. Cl. CPC G01N 33/56983; G01N 33/5308; G01N 33/54313; G01N 33/54386; G01N 33/74; G01N 2333/015; G01N 2333/62; G01N 2469/10

(57) ABSTRACT

The present technology is directed to methods of preparing affinity chromatographic columns using streptavidin-conjugated particles and biotinylated affinity agents (i.e., biotinylated oligonucleotides, biotinylated antibodies or biotinylated antigen-binding fragments thereof). The methods of the present technology can be utilized to perform affinity capture assays in a high-throughput, efficient manner. Further, the affinity chromatographic columns disclosed herein are customizable due to the ability to utilize any biotinylated affinity agent. The methods herein utilize an on-line loading (i.e., loading the column with biotinylated affinity agent via a liquid chromatography system).

### CROSS-REFERENCED TO RELATED APPLICATIONS

This application a continuation of U.S. application Ser. No. 18/647,863, filed Apr. 26, 2024, which claims the benefit of U.S. Provisional Application No. 63/499,094, filed on Apr. 28, 2023, and U.S. Provisional Application No. 63/555,315, filed on Feb. 19, 2024, the entireties of which are incorporated herein by reference.

### FIELD OF THE TECHNOLOGY

The present technology is directed to methods for generating affinity capture columns. More particularly, the present technology is directed to the preparation and use of affinity capture columns with functionalized nonporous particles for use in affinity chromatography.

### BACKGROUND

Chromatography is a separation technique with broad utility across industries, including the pharmaceutical, biotechnology, and chemical industries. Chromatography involves two phases: a stationary phase and a mobile phase. Typically, the stationary phase comprises a porous or nonporous particle that is loaded into a column. The mobile phase then carries a sample through the stationary phase column. One particular application of chromatography, affinity chromatography, utilizes functionalized particles in the stationary phase. The underlying principle behind affinity chromatography is that compounds, or analytes, within a sample will have differential affinities to the functional group on the particle. These differences permit the separation, isolation, and concentration of particular analytes from complex, heterogeneous samples.

Samples generated from laboratory processes (e.g., fermentation), biological sources (e.g., blood), and environmental sources (e.g., wastewater) contain an unpredictable number and composition of analytes. In many instances, only a particular analyte from a given sample is of interest. But due to the complexity of the sample, the analyte of interest can be at such a low concentration, or in the presence of enough impurities, that it is undetectable using standard analytical methods. As such, it is highly desirable to be able to isolate an analyte of interest that is both substantially pure and at high enough concentrations that permit downstream analytical assays.

Current methods of affinity chromatography are hindered by columns that are low throughput and high cost, and often result in loss of analyte(s) of interest due to non-specific binding and/or poor analyte retention. Other types of immunoassays, such as for example, ELISA technology, can be complex, time consuming and highly variable. Therefore, a need in the art exists for affinity chromatography columns that are highly efficient, customizable, reusable, and enable robust separation, collection, and concentration of analytes of interest.

### SUMMARY

In general, the present technology is directed to affinity chromatographic columns for use in affinity chromatography and in affinity capture assays. In particular, the present technology is directed to chromatography columns comprising nonporous particles conjugated to streptavidin, resulting in a streptavidin column. The streptavidin column effectively serves as an immobilized substrate with the capacity to bind any biotinylated molecule, including biotinylated oligonucleotides or biotinylated antibodies and biotinylated antigen-binding fragments thereof. Accordingly, the present technology enables the preparation of an affinity chromatographic column with specificity to an antigen of any antibody or antigen-binding fragment thereof and thus allows the use of biotinylated antibodies or biotinylated antigen-binding fragments thereof to generate customizable affinity chromatographic columns. Further, the present technology enables the preparation of an affinity chromatographic column with specificity to a particular nucleic acid sequence, and thus allows the use of biotinylated oligonucleotides to generate customizable affinity chromatographic columns.

There are many ways to amplify DNA/RNA using PCR, qPCR, dPCR. These methods however do not recognize heterogeneity. In addition, these methods tend to amplify trace DNA/RNA outside of the targeted analyte. Next-Generation Sequencing (NGS) methods can do a better job with heterogeneity but require expensive libraries to complete the analysis. The present technology is directed to a solution that can measure real levels of DNA/RNA analytes used in therapies, as well as any molecular heterogeneity within a short period of time (e.g., <20 minutes, e.g., 10 minutes). The use of ultra-performance streptavidin particles with biotinylated hybridizable sequences for DNA/RNA targets allows for fast separations. The use of advanced detectors (including MS) allows for improved characterization of heterogeneity.

Consequentially, the technology disclosed herein provides a method for creating a customizable affinity chromatographic column to the practitioner's antibody or oligonucleotide of choice. The affinity chromatographic column can be prepared using a single, on-column binding step of the biotinylated oligonucleotide, biotinylated antibody or biotinylated antigen-binding fragment thereof with the streptavidin column of the instant disclosure. Further, the nonporous and rigid form of the underlying streptavidin-conjugated particle enables the use of the affinity chromatographic column under high pressure, while the incredibly strong bond between biotin and streptavidin ensures the column is suitable for use across a range of temperatures, pH, solvents, and sample types.

In one aspect, disclosed herein is a method of loading a biotinylated oligonucleotide, biotinylated antibody or biotinylated antigen-binding fragment thereof to a streptavidin column to form an affinity chromatographic column. The method includes step a) and step b). Step a) is selecting a streptavidin column having a plurality of particles, wherein each particle comprises a nonporous polymer core, a hydrophilic surface on an outer layer of the nonporous polymer core, and one or more molecules of streptavidin conjugated to the hydrophilic surface, wherein the streptavidin has a plurality of accessible binding sites, wherein the particle has an average particle size between 1.0 μm to 10 μm (e.g., 1.5 μm to 8 μm, for example 3.5 μm). Step b) is applying a solution of biotinylated oligonucleotides, biotinylated antibodies or biotinylated antigen-binding fragments thereof to the streptavidin column such that the biotinylated oligonucleotides, biotinylated antibodies or biotinylated antigen-binding fragments thereof bind to a portion of the plurality of accessible binding sites of streptavidin to form the affinity chromatographic column.

In some embodiments, the method further includes step a′), which occurs between step a) and step b). Step a′) comprises washing the streptavidin column prior to applying the solution of biotinylated oligonucleotides, biotinylated antibodies or biotinylated antigen-binding fragments thereof. In some embodiments including step a′), the streptavidin column can be washed with acetonitrile. In certain embodiments including step a′), the streptavidin column cal be washed with a combination of acetonitrile and phosphoric acid. In some embodiments including step a′), the streptavidin column can be washed with sodium phosphate buffer.

Embodiments of the above aspects can include one or more of the following features. In some embodiments, the biotinylated antibodies or biotinylated antigen-binding fragments thereof bind to insulin, AAV9, AAV2, tacrolimus, troponin, IgG, a cytokine, a double-stranded RNA (dsRNA), a host cell protein, or perfluoroalkyl substances (PFAS). In some embodiments, the biotinylated antibodies or antigen-binding fragments thereof are applied to streptavidin column using a high-performance liquid chromatography (HPLC) system, an ultra-high performance liquid chromatography (UHPLC) system, or fast protein liquid chromatography (FPLC) system. In some embodiments, the solution of biotinylated antibodies or biotinylated antigen-binding fragments thereof is applied to the streptavidin column for a time sufficient to bind at least 50% of the plurality of the accessible binding sites of streptavidin. In one embodiment, applying the solution of biotinylated antibodies or biotinylated antigen-binding fragments thereof comprises applying 2.5 μg of biotinylated antibody or biotinylated antigen-binding fragment thereof that binds to insulin to the streptavidin column for 120 sequential injections at 2 minute intervals at a flow rate of 0.1 mL/min. In one embodiment, applying the solution of biotinylated antibodies or biotinylated antigen-binding fragments thereof comprises applying 1.5 μg of biotinylated antibody or biotinylated antigen-binding fragment thereof that binds to AAV9 to the streptavidin column for 70 sequential injections at 2 minute intervals at a flow rate of 0.1 mL/min.

In one aspect, disclosed herein is a method for enriching a target analyte, wherein the method comprises providing the affinity chromatographic column, washing the column with a binding buffer, applying a solution containing the target analyte to the affinity chromatographic column, and washing the column with an elution buffer such that the target analyte is eluted from the column. In some embodiments, the affinity chromatographic column is connected to a high-performance liquid chromatography (HPLC) system, an ultra-high performance liquid chromatography (UHPLC) system, or fast protein liquid chromatography (FPLC) system. In some embodiments, the elution buffer is at least 3 orders of magnitude more acidic than the binding buffer. In some embodiments, the binding buffer has a pH between 7.4-8.0. In some embodiments, the elution buffer has a pH between 1.3-2.3. In some embodiments, the target analyte elutes from the affinity chromatographic column with a peak volume of at least two times more than that of the target analyte eluted from a conventional affinity chromatographic column comprising a plurality of porous particles. In some embodiments, the target analyte elutes from the affinity chromatographic column in less than 1 minute.

In one aspect, the method for enriching a target analyte comprises an additional step of detecting the eluted target analyte with an ultraviolet detector, a fluorescence spectroscopy detector, and/or a mass spectrometry detector. In some embodiments, the method further comprises washing the affinity chromatographic column with an equilibration buffer or the binding buffer. In some embodiments, the method further comprises repeating the steps of washing with a binding buffer, applying the target analyte, washing the column with an elution buffer, and detecting the target analyte with a detector. In some embodiments, the method for enriching a target analyte comprises the additional step of washing the affinity chromatographic column with an equilibration buffer or the binding buffer and repeating the steps of washing with a binding buffer, applying the target analyte, and washing the column with an elution buffer. In some embodiments, the affinity chromatographic column is washed with the equilibration buffer or the binding buffer for less than or equal to 10 minutes.

In some embodiments, the nonporous polymer core comprises divinylbenzene (80). In some embodiments, the coated hydrophilic surface of the nonporous polymer core is selected from the group consisting of: (3-glycidyloxypropyl) trimethoxysilane, (3-glycidyloxypropyl)triethoxysilane, glycidol, glyceroltriglycidyl ether, polyacrylate, and poly(methyl acrylate). In some embodiments, at least a portion of an interior surface of the affinity chromatographic column body is coated with alkylsilyl material. In one embodiment, the alkylsilyl material is a hydrophilic, non-ionic layer of polyethylene glycol silane.

In one aspect, disclosed herein is a method for using an affinity chromatographic column with biotinylated antibodies or biotinylated antigen-binding fragments thereof bound to streptavidin. The method comprises three steps. Step a) comprises selecting an affinity chromatographic column having a plurality of particles, each particle comprising a nonporous polymer core, a hydrophilic surface on an outer layer of the nonporous polymer core, and one or more molecules of streptavidin conjugated to the hydrophilic surface, wherein the streptavidin has a plurality of accessible binding sites, wherein a portion of the plurality of accessible binding sites are bound to a biotinylated antibody or biotinylated antigen-binding fragment thereof, wherein the particle has an average particle size between 1.0 μm to 10 μm (e.g., 1.5 μm to 8 μm). Step b) comprises applying a solution containing a target analyte to the affinity chromatographic column. Step c) comprises washing the column with an elution buffer such that the target analyte is eluted from the column.

In some embodiments, the biotinylated antibodies or biotinylated antigen-binding fragments thereof bind to insulin, AAV9, AAV2, tacrolimus, troponin, IgG, a cytokine, a host cell protein, a dsRNA, or perfluoroalkyl substances (PFAS). In some embodiments, the biotinylated antibodies or antigen-binding fragments thereof are applied to streptavidin column using a high-performance liquid chromatography (HPLC) system, an ultra-high performance liquid chromatography (UHPLC) system, or fast protein liquid chromatography (FPLC) system. In some embodiments, the elution buffer is at least 3 orders of magnitude more acidic than the binding buffer. In some embodiments, the target analyte elutes from the affinity chromatographic column in less than 1 minute. In some embodiments, the method further comprises step d) (which is after step c) washing the column with an equilibration buffer or the binding buffer. In some embodiments, the method further comprises steps b) through c). In some embodiments, the affinity chromatographic column is washed with the equilibration buffer or the binding buffer for less than or equal to 10 minutes.

### BRIEF DESCRIPTION OF THE DRAWINGS

The technology will be more fully understood from the following detailed description taken in conjunction with the accompanying drawings, in which:

FIG. 1A-B shows the nonporous particle functionalized with streptavidin and the affinity chromatography workflow. FIG. 1A provides a representative example of the functionalized particle. FIG. 1B shows the workflow for generating the affinity chromatography column.

FIG. 2 demonstrates the ability for a biotinylated, anti-insulin antibody to bind to a column containing streptavidin-functionalized particles.

FIG. 3A-B shows the affinity capture results from the anti-insulin column. FIG. 3A shows the chromatographs for insulin eluted using affinity capture. FIG. 3B shows the titration curve generated from the affinity capture assay.

FIG. 4A demonstrates the ability for biotinylated, anti-AAV monobody to bind to a column containing streptavidin-functionalized particles in accordance with the present technology.

FIG. 4B-4C demonstrate anti-AAV antibody binding capabilities of two different columns containing streptavidin-functionalized particles in accordance with the present technology.

FIG. 5A-5B shows the affinity capture results from the anti-AAV column. FIG. 5A shows the chromatographs for AAV9 capsid eluted using a step or gradient elution method. FIG. 5B shows the titration curve generated from the affinity capture assay.

FIG. 6 shows the chromatograph for AAV9 using affinity capture with a column utilizing porous 50 μm sized particles (POROS™ Affinity resin available from Thermo Fisher Scientific).

FIG. 7 provides a comparison between an affinity chromatographic column of the present technology using 3.5 μm nonporous polymer particles functionalized with streptavidin bound to a biotinylated anti-AAV antibody and the porous 50 μm sized particles (POROS™ Affinity resin available from Thermo Fisher Scientific).

FIG. 8 demonstrates the ability for a 5′-biotinylated oligonucleotide (dT₂₅) to bind to a column containing streptavidin-functionalized particles in accordance with the present technology.

FIG. 9A-9B depict the binding affinity of poly(A) oligonucleotides (dA) to a dT₂₅ affinity chromatographic column in accordance with the present technology. FIG. 9A shows the chromatograph for the poly(A) oligonucleotides of varying lengths (dA₁₀, dA₁₅, dA₂₀, and dA₃₀). FIG. 9B depicts the chromatography for a different set of lengths (dA₂₋₈, dA₁₀, dA₁₅, and _{dA20-30}.

FIG. 10A-10B depicts the binding affinity of Erythropoietin (EPO) mRNA to a dT₂₅ affinity chromatographic column in accordance with the present technology. FIG. 10A shows the chromatograph for the EPO mRNA at varying concentrations. FIG. 10B shows UV spectra of peaks 1 and 2.

FIG. 11 provides the chromatographs for multiple mRNAs binding to a dT₂₅ affinity chromatographic column in accordance with the present technology.

FIG. 12 provides the chromatographs for mRNA without a poly(A) tail as compared to EPO mRNA and the resultant binding to a dT₂₅ affinity chromatographic column in accordance to the present technology.

FIG. 13A-13B demonstrate the specificity of the dT₂₅ affinity chromatographic column to poly(A) oligonucleotide sequences. FIG. 13A shows size exclusion chromatographs for EPO mRNA samples incubated with various concentrations of dT₁₅₀. FIG. 13B shows dT₂₅ affinity chromatographs for EPO mRNA samples incubated with various concentrations of dT₁₅₀.

FIG. 14A demonstrates streptavidin leaching from a column of the present technology after an applied wash method.

FIG. 14B demonstrates streptavidin leaching from a column of the present using a different wash method.

FIG. 15A-15C demonstrate the binding affinity of dsRNA to an anti-dsRNA affinity chromatographic column in accordance with the present technology. FIG. 15A demonstrates the ability for a biotinylated anti-dsRNA antibody to bind to a column containing streptavidin-functionalized particles. FIG. 15B provides chromatographs of dsRNA eluted from the affinity chromatographic column. FIG. 15C shows the linear correlation between concentration of dsRNA and average peak area.

FIG. 16A-16B demonstrate the effect of biotin endcapping on an affinity chromatographic column. FIG. 16A shows the ability for a biotinylated anti-insulin antibody to bind to a column containing streptavidin-functionalized particles. FIG. 16B shows chromatographs of insulin eluted from a column with or without biotin endcapping.

FIG. 17A-17C demonstrate the binding affinity of host cell proteins to an anti-HCP affinity chromatographic column in accordance with the present technology. FIG. 17A demonstrates the ability for biotinylated anti-HCP polyclonal antibodies to bind to a column containing streptavidin-functionalized particles. FIG. 17B provides chromatographs of HCPs eluted from the affinity chromatographic column. FIG. 17C shows the linear correlation between concentration of HCP and average peak area.

### DETAILED DESCRIPTION

Disclosed herein are methods for preparing affinity chromatography columns and their uses thereof. In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means ±5%. It is also to be noted that as used herein and in the claims, the singular forms “a,” “and” and “the” include plural references unless the context clearly dictates otherwise.

### Definitions

As used herein, the terms “affinity capture chromatography” and “affinity chromatography” are used interchangeably unless defined otherwise.

As used herein, the term “conjugate” refers to a compound formed by the chemical bonding of a reactive functional group of one molecule, such as streptavidin, with an appropriately reactive functional group of another molecule, such as an epoxide. An example of suitably reactive functional groups is a nucleophile/electrophile pair. For instance, the nucleophile may be an amine group from an amino acid of streptavidin, and the electrophile is an epoxide.

As used herein, the term “conjugated” refers to the linkage of two molecules formed by the chemical bonding of a reactive functional group of one molecule, such as streptavidin, with an appropriately reactive functional group of another molecule, such as an epoxide.

As used herein, the term “non-specific binding” refers to the binding of unintended compounds to the antibody or antigen-binding fragment thereof.

As used herein, the term “functionalized” refers to a particle that comprises one or more protein conjugated to its surface. The protein may be an antibody or antigen-binding fragment thereof.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)₂, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)₂ fragments refer to antibody fragments that lack the Fc portion of an intact antibody.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein, the term “bispecific antibody” refers to an antibody that is capable of binding at least two different antigens.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

The term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Unless defined otherwise, all technical and scientific terms used herein have the same meaning as commonly understood by one of ordinary skill in the art to which this disclosure belongs.

The streptavidin column of the instant disclosure effectively serves as an immobilized substrate with the capacity to bind any biotinylated molecule, including biotinylated oligonucleotides, biotinylated antibodies and biotinylated antigen-binding fragments thereof. As such, the affinity chromatographic column can be prepared using a single, on-column binding step of the biotinylated oligonucleotide, or biotinylated antibody or biotinylated antigen-binding fragment thereof with the streptavidin column. Without wishing to be bound by theory, it is believed that any antibody can be biotinylated. Thus, the present technology provides methods for generating an affinity chromatographic column to the antigen of any antibody or the complementary base pairs of any oligonucleotide.

Typically, affinity chromatography involves the separation of target analytes from a complex, heterogeneous mixture such that the target analyte can be measured, quantified, collected, and/or monitored in an analytical assay or subsequent procedure. Methods for capturing target analytes can be tedious and burdensome, and frequently rely on the chemical modification or alteration of the analyte such that it can be captured using affinity chromatography. For instance, if a target analyte is a protein, it would require the chemical modification of that protein with an affinity tag, such as a polyhistidine tag, such that it could be captured on an anti-histidine column. This chemical modification has the potential to disrupt native protein function and/or require significant assay optimization on an application by application basis. Alternatively, one must rely on non-targeted chromatography methods, such as size exclusion chromatography or ion-exchange chromatography, which rely on intrinsic properties of the target analyte that potentially overlap with, or are shared between, other compounds in the heterogeneous mixture. To circumvent the aforementioned problems, disclosed herein are affinity chromatographic columns that rely on the highly specific binding domains of antibodies to capture target analytes. The affinity chromatography columns disclosed herein are customizable and can utilize any biotinylated antibody or biotinylated antigen-binding fragment thereof. In one aspect, these columns are packed with nonporous particles. The nonporous particles comprise a nonporous polymer core, a hydrophilic surface on an outer layer of the nonporous polymer core, and one or more molecules of streptavidin conjugated to the hydrophilic surface, thereby forming streptavidin-functionalized particles for use within a column.

### Particles for Use in Preparing the Streptavidin Column

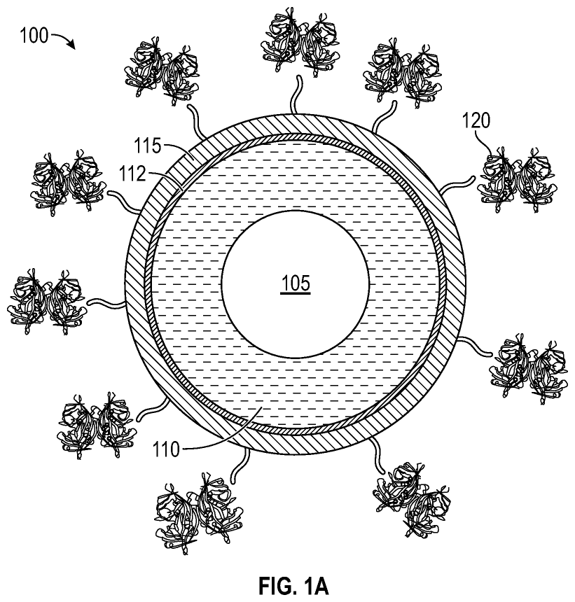

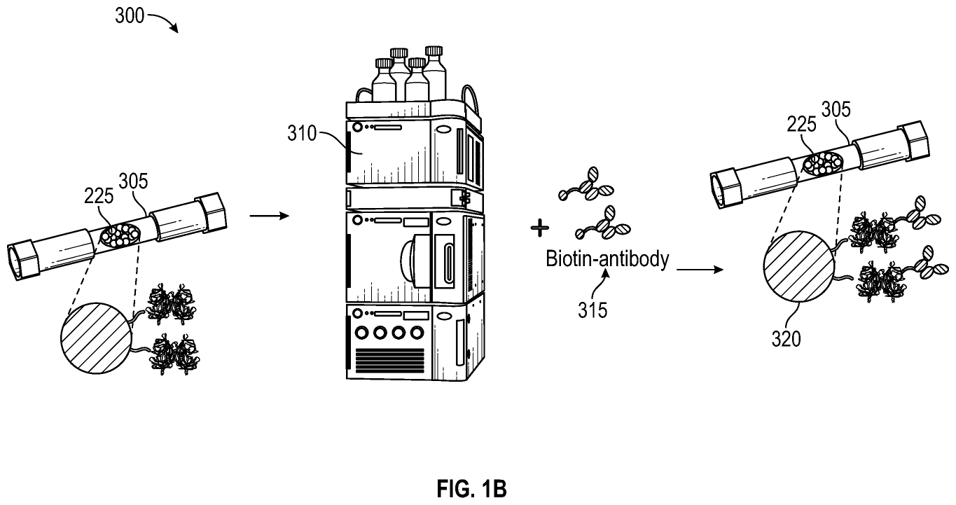

FIG. 1A illustrates an embodiment of a streptavidin-functionalize particle having a nonporous polymer core in accordance with the present technology. The particle illustrated in FIG. 1A has a form factor (e.g., highly spherical, nonporous, and rigid) to withstand operating conditions of HPLC and UHPLC. Particle 100 includes a nonporous polymer core 112 having an inner core region 105 and a radially extending region 110 surrounding the inner core region 105. The inner core region 105 typically is formed of a polymer or a homogenous blend of polymers, whereas the radially extending region 110 typically is formed of two or more polymers to form a gradient within this region. For example, core region 105 can be formed of polystyrene, whereas radially extending region 110 contains a gradient composition transitioning from 100% polystyrene to 80% to 100% DVB with any remainder being polystyrene. In some embodiments, the nonporous particle is highly spherical with a smooth surface. In some embodiments, the nonporous particle is highly spherical with a bumpy, convex surface.

In one embodiment, to form nonporous polymer cores 112, the following three steps were used. In step one: 561.1 g of reagent alcohol (90% ethanol, ^{˜}5% methanol and ^{˜}5% isopropanol), 16.9 g of polyvinylpyrrolidone (PVP-40, average molecular weight 40,000), 1.6 g of 2,2′-Azobis (2-methylpropionitrile) (AIBN), 6.7 g of Triton™ N-57, 80.1 g of styrene and 2.4 g of poly(propylene glycol) dimethacrylate (average molecular weight 560) were charged into a reactor. After purging with nitrogen, the reaction mixture was heated to 70° C. with stirring and was held at 70° C. until the completion of all the reaction steps. In step two: after the step one reaction mixture was held at 70° C. for 3 hours, a solution containing 52.0 g of DVB 80, 24.0 g of styrene, 51.0 g of PVP-40, 1080.4 g of reagent alcohol (90% ethanol, ^{˜}5% methanol and ^{˜}5% isopropanol) and 54.1 g of p-xylene was added to the reaction mixture at a constant flow rate over two hours. In step three: after the completion of solution charge in step two, a primer coating solution containing 31.2 g of glycidyl methacrylate (GMA), 6.2 g of ethylene glycol dimethacrylate (EDMA), 12.9 g of PVP-40 and 381.9 g of reagent alcohol (90% ethanol, ^{˜}5% methanol and ^{˜}5% isopropanol) was added to the reaction mixture at a constant flow rate over 1.5 hours. After the reaction mixture was held at 70° C. for a total of 20 hours, the particles were separated from the reaction slurry by filtration. The particles were then washed with methanol, followed by tetrahydrofuran (THF), and followed by acetone. The final product was dried in vacuum oven at 45° C. overnight. 91.8 g of monodisperse 2.3 μm polymer particles were obtained.

While the embodiment shown in FIG. 1A illustrates that the nonporous polymer core 112 has two regions (the inner core region 105 and the radially extending region 110), that need not be the case. Other embodiments may feature a nonporous polymer core having a singular region, i.e., the nonporous polymer core extends from the center of the particle to an outer surface of the nonporous polymer core 112. Other embodiments of nonporous polymer cores and particles suitable for use with the present technology are described in U.S. Patent Publication No. 2019/0322783.

As illustrated in FIG. 1A, a hydrophilic surface or layer 115 is formed on an outer surface (i.e., opposite to the center region 105) of the nonporous polymer core. In one embodiment, the hydrophilic surface 115 is formed through the application of a hydrophilic primer coating solution containing 36.2 g of glycidyl methacrylate (GMA), 7.44 g of ethylene glycol dimthacrylate (EDMA), 8.21 g of PVP360(PVP360, average molecular weight 360,000) and 489.4 g of reagent alcohol (90% ethanol, ^{˜}5% methanol and ^{˜}5% isopropanol). This solution was added into to a mixture containing the nonporous polymer cores at a constant flow rate over about 1.5 hours to form hydrophilic surface 115.

The above example is provided for illustration purposes only. Other types of hydrophilic surfaces can be applied. For example the hydrophilic layer may also be formed of (3-glycidyloxypropyl) trimethoxysilane, (3-glycidyloxypropyl) triethoxysilane, polyacrylate, and/or poly(methyl acrylate), glycidol, glyceroltriglycidyl ether, or any other type of hydrophilic material.

To form streptavidin-functionalized particles, a linker is used to conjugate the hydrophilic surface 115 to the streptavidin. Referring to FIG. 1A, particle 100 results from the attachment of streptavidin to the hydrophilic surface through use of the linker. In one embodiment, the streptavidin 120 is attached to the hydrophilic surface 115 by a ring opening of a surface epoxide. That is, an epoxy linker is utilized. That need not be the case. Other linkages can be used. For example, instead of attaching using an epoxy linker, attachment can be made using an amide bond formation, a cyanogen bromide reaction, or aldehyde condensation. One of ordinary skill in the art would understood that a number of linkers are suitable for use with the present technology.

In some embodiments, the epoxy linker has a formula:

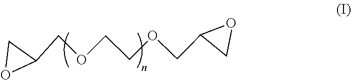

wherein n (the number of ethylene oxide repeating units) is an integer from 1 to 150. In some embodiments, n is 1, 2, 3, 4, 5, 6, 7, 8, 9, 10, 11, or 12. In some embodiments, n is 1, 4, or 9. In some embodiments, n is 1.

### Methods of Making Affinity Chromatographic Columns

In one aspect, the nonporous particles are conjugated to one or more molecules of streptavidin. In some embodiments, the streptavidin molecules are present at 1-5 μg/mg of particle. In some embodiments, the streptavidin molecules are present at 1-2, 2-3, 3-4, or 4-5 μg/mg of particle. In some embodiments, the nonporous particles are conjugated to avidin. In some embodiments, the nonporous particles are conjugated to a protein that binds to biotin. One of ordinary skill in the art would understand that the use of avidin or streptavidin would achieve similar results. The nonporous particles conjugated to streptavidin are then suitable for use in preparing affinity chromatographic columns with any biotinylated antibody or biotinylated antigen-binding fragment thereof.

FIG. 1B shows the process of preparing the affinity chromatographic column, wherein the column is first packed with the streptavidin-conjugated particles to provide the streptavidin column (step a). This is then connected to a liquid chromatography device. The biotinylated affinity agent (for example the biotinylated antibody or biotinylated antigen-binding fragment thereof) is then flowed on the column, wherein the biotinylated affinity agent binds to one or more accessible streptavidin binding sites (step b).

As used herein, the term “biotinylated affinity agent” refers to a biotinylated molecule that can specifically bind to a target antigen or complementary nucleic acid sequence. The biotinylated affinity agent may be a biotinylated antibody or antigen-binding fragment thereof or a biotinylated oligonucleotide. The preparation of biotinylated molecules is a process well known and understood in the art. In some embodiments, the molecule is biotinylated with a biotin derivative, including but not limited to iminobiotin, desthiobiotin, disulfide biotin azide, disulfide biotin alkyne or other biotin derivatives.

An advantage of the method of pumping the biotinylated affinity agent across a bed of particles packed into a device includes precise metering of reagents, contact times and ability to use post column detectors (e.g., use of detector to monitor amount of biotinylated molecule eluting from column versus loading on the column).

The pump system used to pump fluids across the plurality of particles in the chromatographic devices include UHPLC system pumps, HPLC system pumps, and FPLC system pumps. These pump-column systems can be connected to a post-column detector (UV, TUV, PDA, RI, MALS, MS, FL) or they can flow without attachment to a detector. Multiple columns can be coupled in series or in parallel using tubing to increase throughput. The effluent of the columns can be isolated and reused or directed to suitable waste container.

After flowing the solution of molecules (i.e., biotinylated affinity agent) through the plurality of particles packed in the column, the chromatographic device can be washed with water, PBS buffer or storage buffer, and then stoppered or enclosed to prevent evaporation and, if desired, stored in a refrigerator until ready for use.

In some embodiments, a column packed with a plurality of functionalized streptavidin particles can be washed with water, a buffer or storage solution, and/or an acetonitrile-based solution (e.g., 20% acetonitrile and 1% phosphoric acid) prior to adding the solution containing biotinylated affinity agent. The column packed with the plurality of functionalized streptavidin particles can be stored prior to the loading of the biotinylated affinity agent. That is between step a and step b of the method shown in FIG. 1B.

In one embodiment, the chromatography column is prepared as follows. The nonporous particles functionalized with streptavidin are packed into a chromatography column, resulting in a streptavidin column. A number of column sizes and materials are suitable for use in the methods disclosed herein. In some embodiments, the column material is stainless steel, polyetheretherketone (PEEK) lined steel, titanium, or a stainless steel alloy such as MP35n. In some embodiments, the column has an internal diameter ranging from 75 μm to 4.6 mm. In some embodiments, the column has a length between 5 to 300 mm. In a preferred embodiment, the column has an internal diameter between 1 to 2 mm and a length between 5 to 20 mm. The column surface can be unmodified or modified to generate a high-performance surface. Chromatography columns suitable for use with the methods disclosed herein are compatible with any standard liquid chromatography system, including high-performance liquid chromatography (HPLC) systems, ultra-high performance liquid chromatography (UHPLC) systems, and fast protein liquid chromatography (FPLC) systems.

In some embodiments, the liquid chromatography system is connected in series to a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series.

In some embodiments, the interior surfaces of the column are treated to reduce non-specific binding and enhance overall efficiency of the liquid chromatography system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevent or minimize contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl)ethane or bis(trimethoxysilyl)ethane. Other embodiments of alkylsilyl coatings suitable for use with the present technology are described in U.S. Patent Publication No. 2019/0086371 and U.S. Application Publication No. 2022/0118443.

The streptavidin column, comprising the streptavidin-functionalized particles, is then connected to a suitable liquid chromatography system. The biotinylated antibody or biotinylated antigen-binding fragments thereof are flowed through the column, permitting the ionic binding of the biotin group to one of the accessible streptavidin binding sites. Streptavidin naturally occurs as a homo-tetramer with four available binding pockets for biotin. Due to the stochastic nature of how streptavidin is conjugated to the nonporous particle, a given streptavidin molecule may have 0, 1, 2, 3, or 4 accessible binding sites. In some embodiments, the biotinylated affinity agent may bind to 0, 1, 2, 3, or 4 accessible binding sites of a given streptavidin molecule. In some embodiments, the biotinylated antibodies or biotinylated antigen-binding fragments thereof may bind to 0, 1, 2, 3, or 4 accessible binding sites of a given streptavidin molecule. In some embodiments, the biotinylated oligonucleotide may bind to 0, 1, 2, 3, or 4 accessible binding sites of a given streptavidin molecule. In some embodiments, a portion of the plurality of the accessible streptavidin binding sites are occupied by a biotinylated oligonucleotide or a biotinylated antibody or antigen-binding fragment thereof. In some embodiments, 50-55%, 55-60%, 60-65%, 65-70%, 70-75%, or more than 75% of the plurality of the accessible streptavidin binding sites are occupied by a biotinylated oligonucleotide, or a biotinylated antibody or biotinylated antigen-binding fragment thereof. The extremely strong affinity between biotin and streptavidin ensures that the biotinylated antibodies or antigen-binding fragments thereof are immobilized onto the solid phase of the chromatography column. The interaction between biotin and streptavidin is resistant to organic solvents, changes in pH, changes in temperature, detergents, and many concentrations of denaturants. As such, the affinity chromatographic columns disclosed herein are suitable for use with a range of organic solvents, pH, temperatures, and samples.

To increase the selectivity of the affinity chromatographic column, it is desirable to maximize the percentage of accessible binding sites of the streptavidin molecules present in the streptavidin column that are bound with a biotinylated oligonucleotide, or a biotinylated antibody or biotinylated antigen-binding fragment thereof. The percentage of accessible binding sites that are occupied, or the extent of saturation, can be determined by monitoring the effluent while preparing the affinity chromatographic column. When initially applying the biotinylated affinity agent (such as a biotinylated oligonucleotide, or a biotinylated antibody or biotinylated antigen-binding fragment thereof) to the streptavidin column, the biotinylated affinity agent will bind to any accessible streptavidin site. As such, upon initial application of biotinylated affinity agent, the biotinylated affinity agent thereof should be present at low levels in the effluent as measured by a detector. In some embodiments, the detector used to monitor the effluent is an ultraviolet spectroscopy (UV) detector. In some embodiments, the detector used to monitor the effluent is a fluorescence spectroscopy detector. As the streptavidin column increases in saturation, the amount of biotinylated affinity agent will increase in the effluent. The increase in concentration of the biotinylated affinity agent thereof indicates an increase in the saturation of accessible binding sites of the streptavidin column. If the effluent is monitored over time, the increase in biotinylated affinity agent present in the effluent will plateau, indicating saturation of the streptavidin column.

In some embodiments, it may be advantageous to endcap the plurality of particles with excess biotin. This process, referred to herein as ‘biotin endcapping’ involves the addition of free biotin to the plurality of particles after a biotinylated affinity group is added. The free biotin can interact with remaining, unoccupied binding sites on the streptavidin molecules, increasing efficiency and reducing noise. Said unoccupied streptavidin binding sites may be present due to, for example, incomplete saturation of the column or due to steric hindrance that blocks the biotin of the affinity group from binding to streptavidin. Example 15 describes a method of biotin endcapping.

Example 3 provides a method of loading a streptavidin column with a biotinylated antibody that binds to insulin. As the biotinylated antibody is loaded onto the streptavidin column, the effluent is monitored using a UV detector.

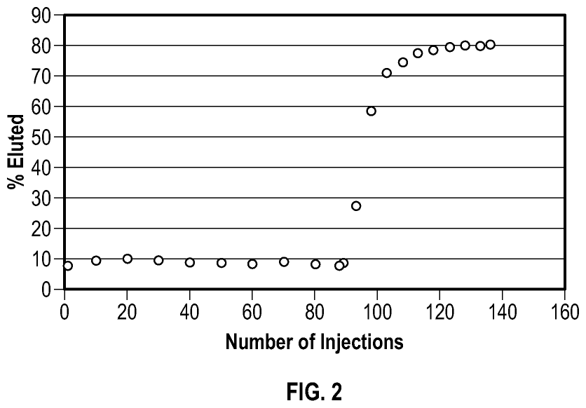

FIG. 2 plots the percentage of biotinylated antibody from Example 3 present in the effluent over the duration of the column loading. After ˜90 injections, the percentage of biotinylated antibody present in the effluent increases, indicating the streptavidin column is beginning to reach saturation. After ˜120 injections, the increase in biotinylated antibody present in the effluent plateaus, meaning that the plurality of accessible binding sites of streptavidin in the column is saturated.

Similarly, Example 5 provides a method of loading a streptavidin column with a biotinylated antibody that binds to AAV capsids. As the biotinylated antibody is loaded onto the streptavidin column, the effluent is monitored using a UV detector.

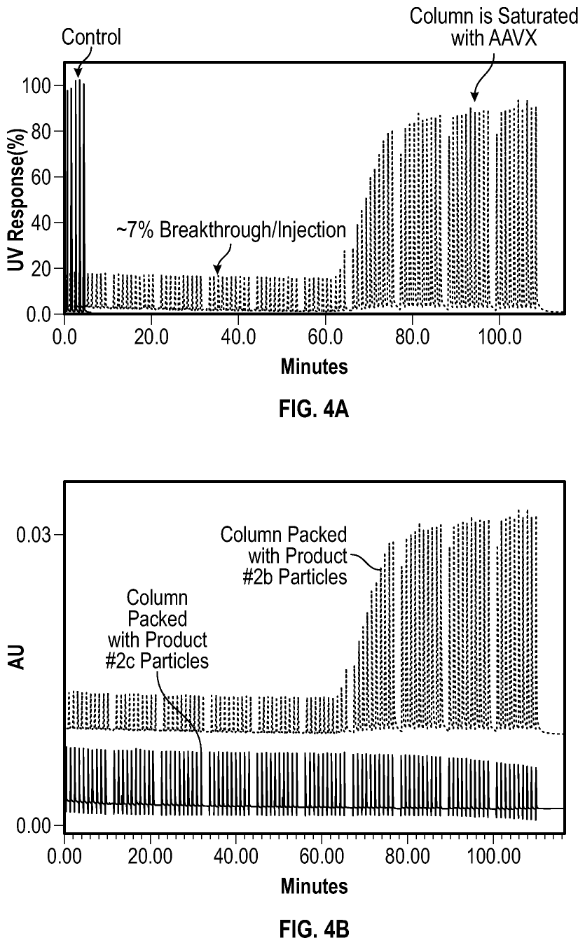

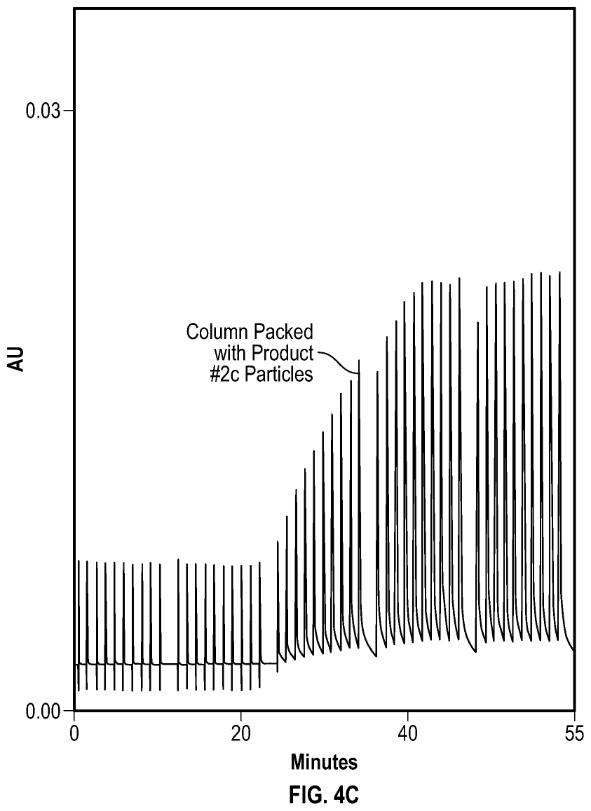

FIG. 4 plots the percentage of biotinylated antibody from Example 5 present in the effluent over the duration of the column loading. After ˜60 injections, the percentage of biotinylated antibody present in the effluent increases, indicating the streptavidin column is beginning to reach saturation. After ˜100 injections, the increase in biotinylated antibody present in the effluent plateaus, meaning the plurality of accessible binding sites of streptavidin in the column is saturated.

It is expected that a portion of the biotinylated affinity agent will be present in the effluent during loading due to breakthrough resulting from the stochastic binding process. In some embodiments, approximately 0-10% of the biotinylated affinity agent will be present in the effluent prior to saturation. In some embodiments, approximately 0-2%, 2-4%, 4-6%, 6-8%, or 8-10% of the biotinylated affinity agent will be present in the effluent prior to saturation.

The resultant column, wherein a plurality of the accessible binding sites of the streptavidin molecules are bound to a biotinylated affinity agent, can subsequently be used for affinity chromatography.

### Antibodies Suitable for Use in Preparing Affinity Chromatographic Columns

A number of affinity groups are suitable for use in the disclosed technology, provided that the affinity groups are biotinylated. In some embodiments, the affinity group is a biotinylated antibody or biotinylated antigen-binding fragment thereof. In some embodiments the biotinylated antibody is a polyclonal antibody, monoclonal antibody, nanobody, monobody, single domain antibody, bispecific antibody, or camelid. In some embodiments, the biotinylated antibody may be of an IgG, IgM, IgA, IgE, or IgD isotype. In some embodiments, the biotinylated antibody may be derived from a human, mouse, rabbit, goat, or other species. In some embodiments, the antibody may be a chimeric antibody. In some embodiments, the antibody may be a humanized antibody. In one aspect, any protein-based affinity group that is biotinylated is suitable for use with the present technology.

In some embodiments, the affinity group is a biotinylated anti-insulin antibody. In some embodiments, the affinity group is a biotinylated anti-AAV9 antibody. In some embodiments, the affinity group is a biotinylated anti-AAV2 antibody. In some embodiments, the affinity group is a biotinylated anti-AAV capsid antibody, wherein the anti-AAV capsid antibody has affinity to different AAV serotypes, including AAV1, AAV2, AAV3, AAV4, AAV5, AAV6, AAV7, AAV8, and AAV9. In some embodiments, the affinity group is a biotinylated anti-PFAS (perfluoroalkyl substance) antibody. In some embodiments, the affinity group is a biotinylated polyclonal antibody (or a population of biotinylated polyclonal antibodies) that bind to host cell proteins (a biotinylated anti-HCP antibody). In some embodiments, the affinity group is a biotinylated anti-IgG antibody. In some embodiments, the affinity group is a biotinylated anti-dsRNA antibody.

As used herein, the term “host cell protein” refers to process-related proteinaceous impurities present in a host cell culture or host cell line. Thus, a host cell protein may be any protein present in a host cell culture or a host cell line.

### Oligonucleotides Suitable for Use in Preparing Affinity Chromatographic Columns

In yet other embodiments, the affinity group is a biotinylated oligonucleotide (or oligomer, used interchangeably herein). The oligonucleotide can be biotinylated on either the 5′ or 3′ end. The oligonucleotide can range from 5-50 nucleotides. In some embodiments, the oligonucleotide comprises 25 nucleotides. Any or all of the nucleotides in the oligonucleotide can further be modified using methods known in the art. The oligonucleotide may comprise deoxyribonucleic acids (DNA), ribonucleic acids (RNA), or a combination thereof.

Biotinylated oligonucleotides suitable for use in the present technology generally follow the formula:

B₀₋₁(Xₙ)ₚB′₀₋₁ (Formula II)

wherein B and B′ are independently a biotin group, provided that at least one of B or B′ is present;
X is independently a nucleotide, nucleoside, or derivative thereof, including but not limited to adenosine, thymidine, guanosine, and cytidine;
n is independently 1-50; and
p is independently 1-50.

In some embodiments of the above formula, B is 1, X is thymidine, n is 25, p is 1, and B′ is 0. The resultant 5′ biotinylated oligonucleotide comprises 25 thymidine units (i.e., a 25-mer of thymidine or dT₂₅).

In some embodiments, the affinity group is a biotinylated oligonucleotide of Formula II. In some embodiments, the biotinylated oligonucleotide sequence is complementary to a target analyte sequence.

In one aspect, any nucleic acid-based affinity group that is biotinylated is suitable for use with the present technology. In some embodiments, the affinity group is a biotinylated oligonucleotide. The oligonucleotide may comprise deoxyribonucleic acids (DNA), ribonucleic acids (RNA), or a combination thereof. DNA oligonucleotides comprise the nucleotides cytidine, guanosine, adenosine, and thymidine. RNA oligonucleotides comprise the nucleotides cytidine, guanosine, adenosine, and uridine. In some embodiments, the oligonucleotide may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Oligonucleotides may further be modified at the nucleobase, sugar, or phosphodiester backbone with an array of chemical modifications which are further reviewed in Epple et al., Emerg. Top. Life. Sci. (2021) 5(5):691-697. Methods of biotinylating oligonucleotides, including modified oligonucleotides or oligonucleotides comprising non-naturally occurring nucleic acid analogues, are well known in the art and would be readily understood by a person of ordinary skill.

Antibodies suitable for use with the disclosed technology can be generated by any method known in the art. For example, antibodies can be generated using hybridoma technology. Oligonucleotides suitable for use with the disclosed technology can be generated by any methods known in the art. For example, oligonucleotides can be synthesized using solid-phase synthesis. Oligonucleotides, antibodies or antigen-binding fragments thereof can be biotinylated by any means known in the art. For example, the antibody or antigen-binding fragment thereof can be biotinylated using commercially available kits, conjugation methods, or enzymatic methods.

### Methods of Using Affinity Chromatography Columns

The resultant affinity chromatographic column of the disclosed technology can be used to perform affinity capture of target analytes with affinity to the biotinylated antibody or biotinylated antigen-binding fragment which was used to make the column.

The affinity chromatographic column is suitable for use in conjunction with any liquid chromatography system as described above. In some embodiments, the liquid chromatography system is an HPLC, UHPLC, or FPLC.

Example 4 describes a method of performing an affinity capture assay using an affinity chromatographic column prepared with a biotinylated anti-insulin antibody. The affinity chromatographic column was used to bind and elute purified insulin at a range of concentrations.

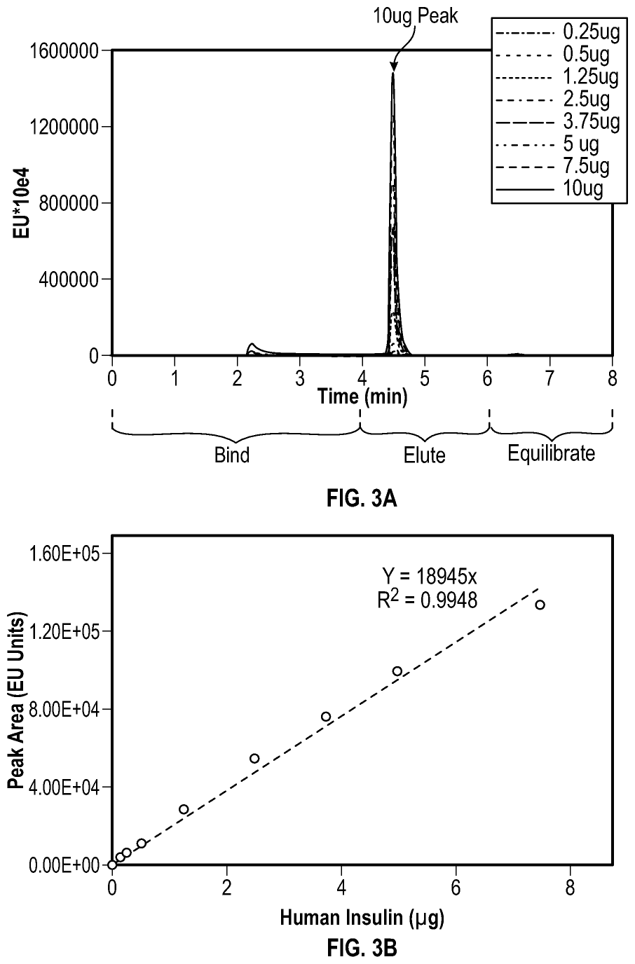

FIG. 3A shows exemplary results from Example 4, wherein the insulin was efficiently captured by the affinity chromatographic column and eluted. The insulin eluted in less than 1 minute, with sharp, narrow peaks that permit for robust quantification and titration analysis as shown in FIG. 3B.

Example 6 describes a method of performing an affinity capture assay using an affinity chromatographic column prepared with a biotinylated anti-AAV antibody. The affinity chromatographic column was used to bind and elute AAV9 capsids at a range of concentrations.

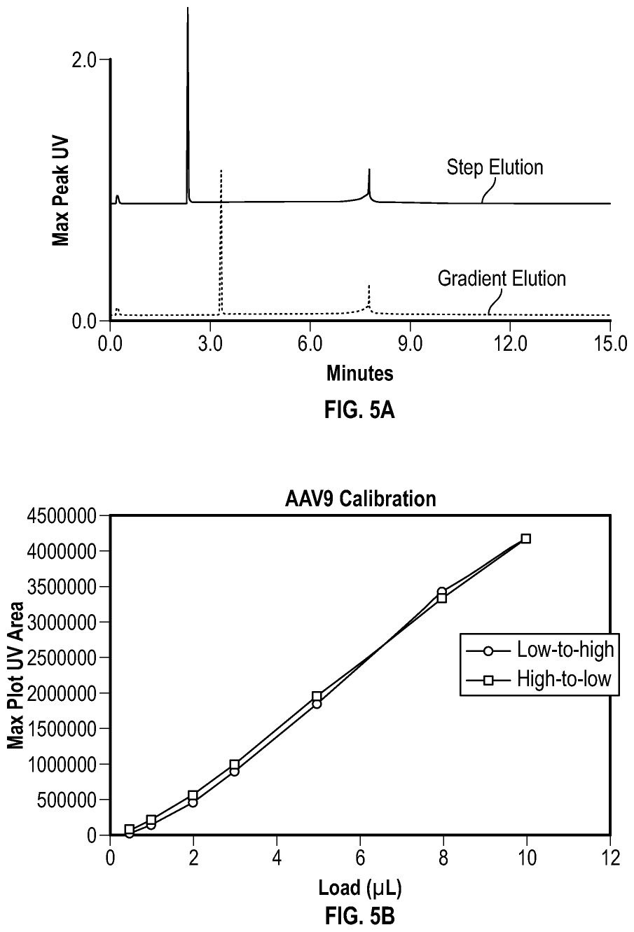

FIG. 5A shows exemplary results from Example 6, wherein the AAV9 capsids were efficiently captured by the affinity chromatographic column and eluted. The AAV9 capsids eluted with sharp peaks that permitted quantification and titration analysis at a range of concentrations as shown in FIG. 5B.

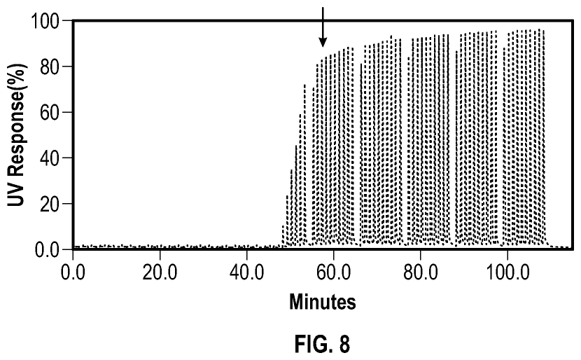

Example 9 describes a method of performing an affinity capture using an affinity chromatographic column prepared with a biotinylated dT₂₅ oligonucleotide. FIG. 8 shows exemplary results from the preparation of the dT₂₅ column of Example 9.

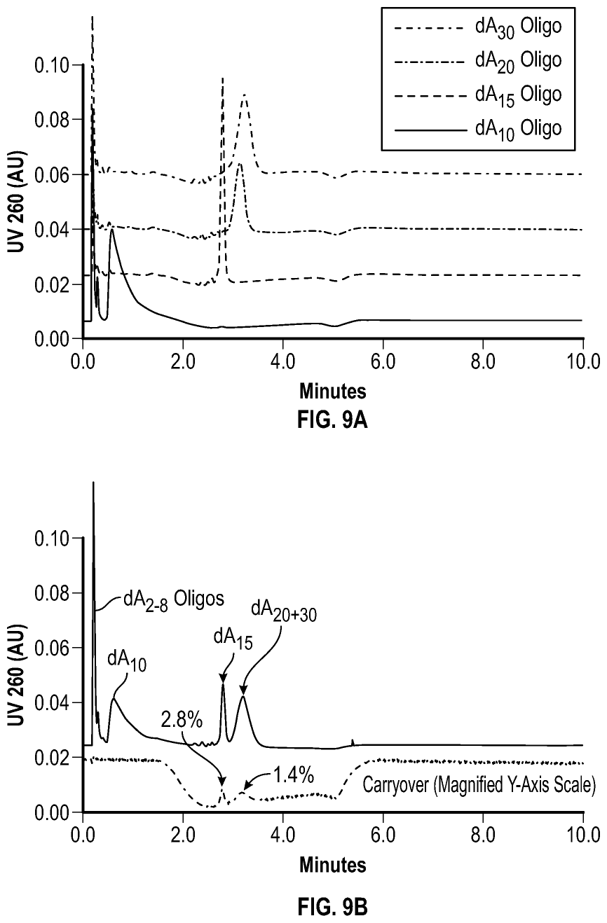

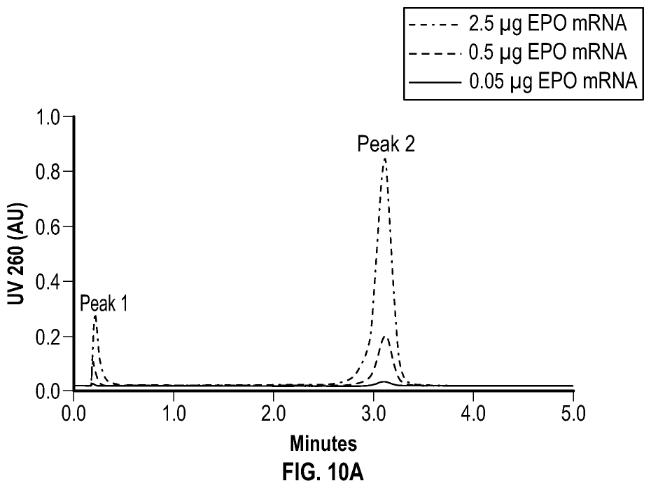

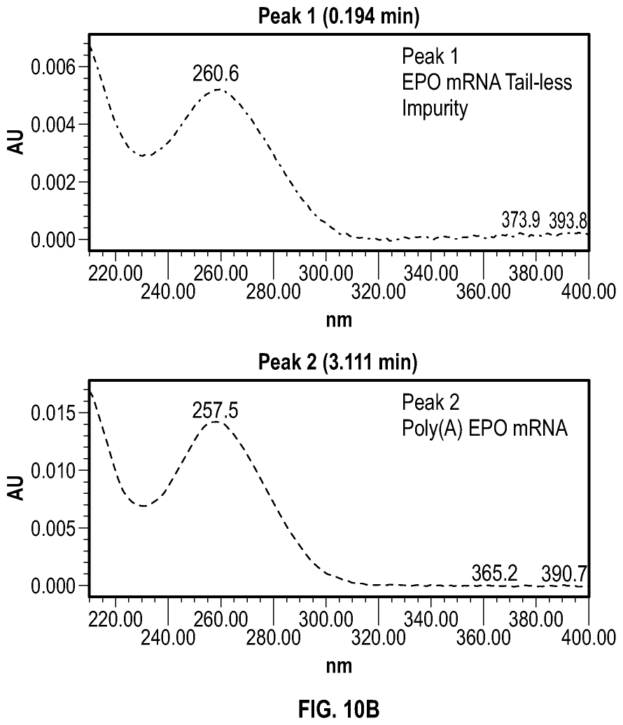

Example 10 describes a method of performing an affinity capture using an affinity chromatographic column prepared with a biotinylated dT₂₅ oligonucleotide using an array of sample oligonucleotides, including poly(A) oligonucleotides (FIG. 9A and 9B), Erythropoietin (EPO) mRNA (FIGS. 10A and 10B), Firefly luciferase Fluc mRNA (FIG. 11), Cas9 mRNA (FIG. 11), and FlucBeta mRNA (FIG. 11).

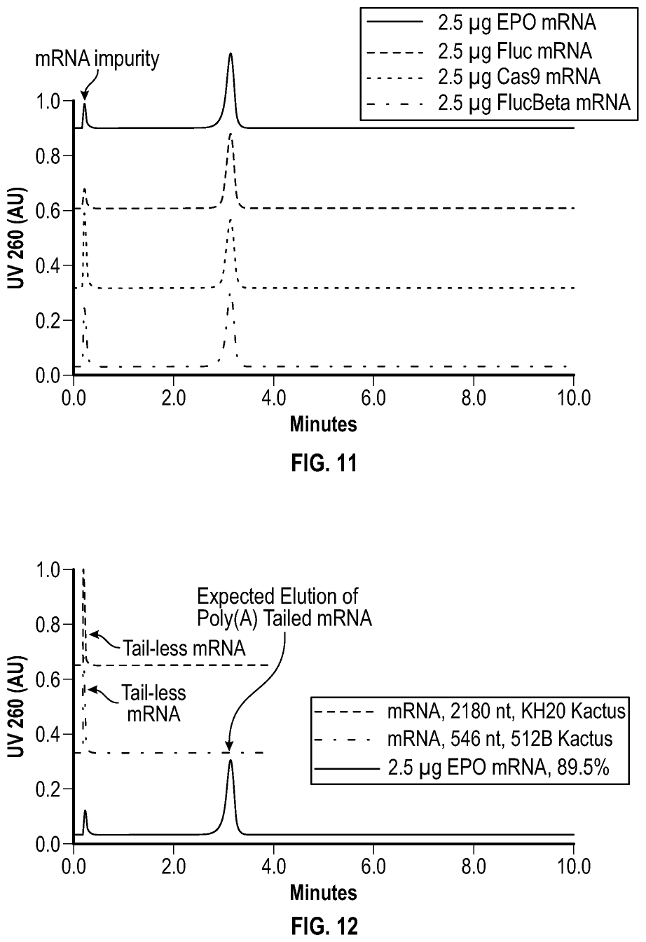

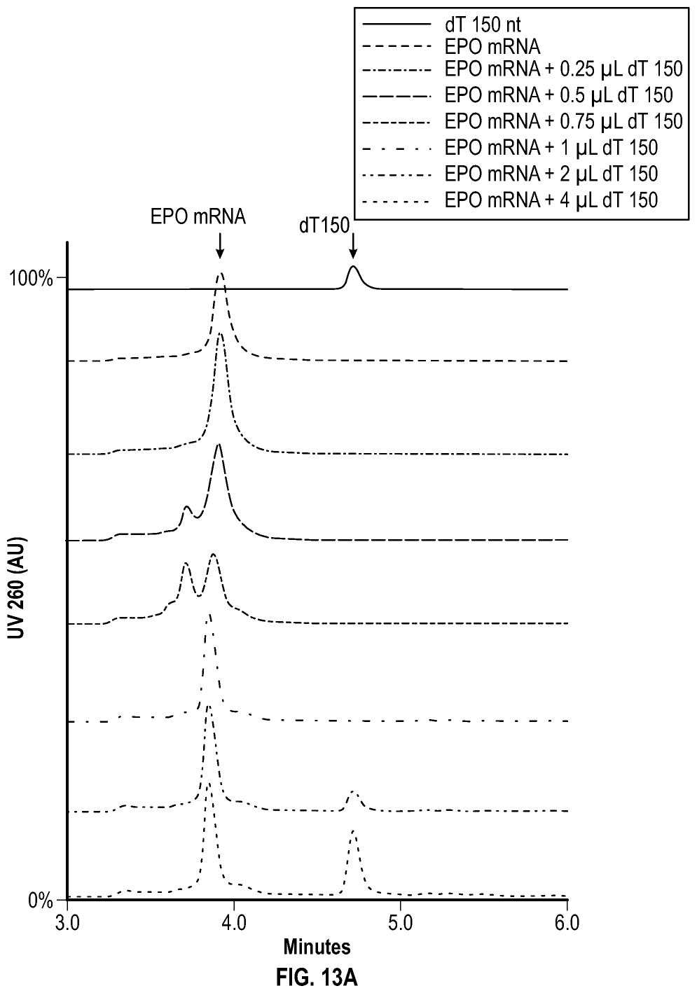

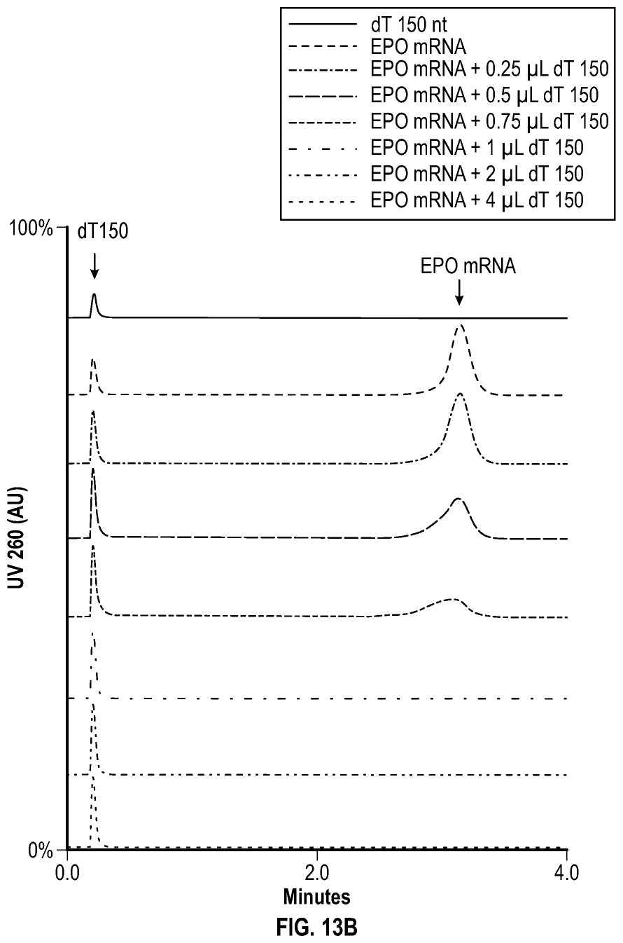

FIG. 12 demonstrates that the dT₂₅ column of the instant technology specifically binds to oligonucleotides with a poly(A) sequence, such as the poly(A) tail of mRNA. FIG. 13 provides further evidence to this specificity: EPO mRNA that is hybridized to a dT₁₅₀ oligonucleotide (i.e., the poly(A) tail is no longer accessible) is not retained by the dT₂₅ column.

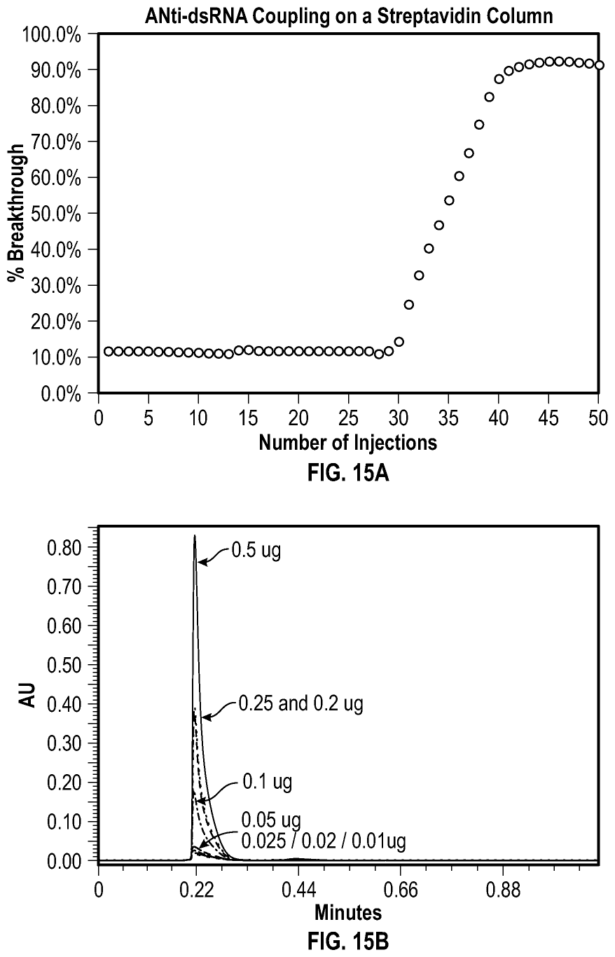

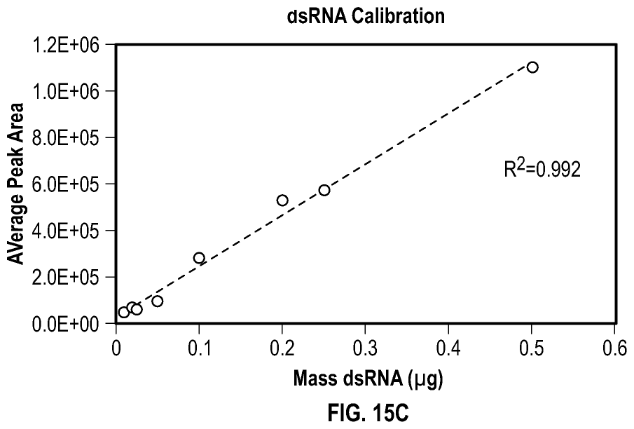

Example 13 and FIG. 15A describes a method of generating an affinity chromatographic column with a biotinylated anti-dsRNA. Example 14 and FIGS. 15B-15C describes a method of performing an affinity capture of dsRNA using the affinity chromatographic column of Example 13.

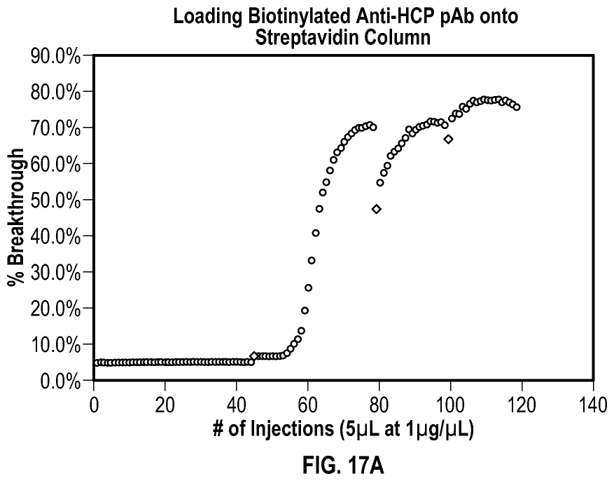

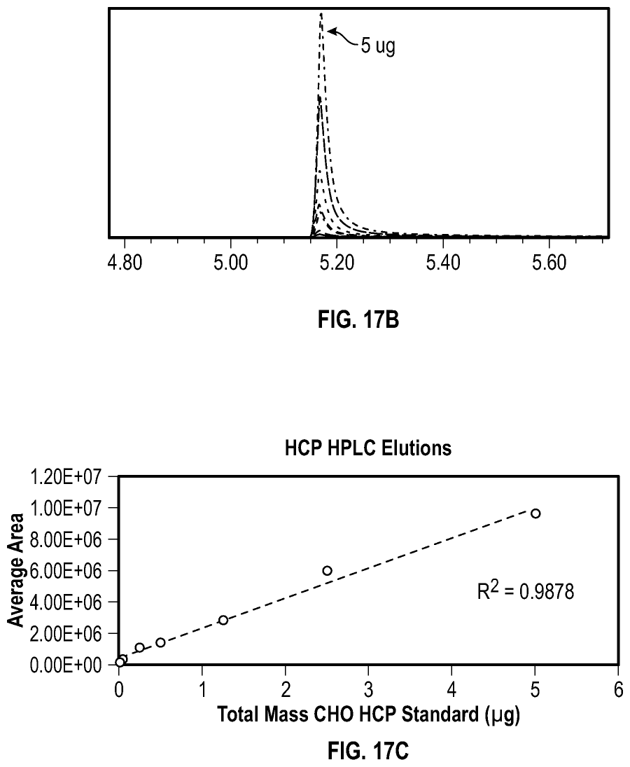

Example 16 and FIG. 17A describe a method of generating an affinity chromatographic column with biotinylated polyclonal anti-HCP antibodies. Example 17 and FIGS. 17B-17C describe a method of performing an affinity capture of HCPs using the affinity chromatographic column of Example 16.

Buffers suitable for use in liquid chromatography are well known in the art. It is understood that a range of binding buffers are suitable for use with the disclosed technology and a person of ordinary skill in the art could determine without undue experimentation the appropriate binding buffer that is suitable with a given target analyte. In one aspect, the binding buffer has a pH between 7.0 and 8.0. In some embodiments, the pH of the binding buffer is between 7.0-7.1, 7.1-7.2, 7.2-7.3, 7.3-7.4, 7.4-7.5, 7.5-7.6, 7.6-7.7, 7.7-7.8, 7.8-7.9, or 7.9-8.0. In one embodiment, the pH of the binding buffer is 7.3. In one embodiment, the pH of the binding buffer is 7.4. In one aspect, the elution buffer has a pH between 1.0 and 3.0. In some embodiments, the pH of the elution buffer is between 1.0-1.2, 1.2-1.4, 1.4-1.6, 1.6-1.8, 1.8-2.0, 2.0-2.2, 2.2-2.4, 2.4-2.6, 2.6-2.8, or 2.8-3.0. In one embodiment, the pH of the elution buffer is 1.3. In one embodiment, the pH of the elution buffer is 2.3. It is important that the pH of the elution buffer be at least 3 magnitudes more acidic than the binding buffer.

In some embodiments pertaining to biotinylated oligonucleotides, a sample may be eluted by altering the ionic strength of the elution buffer (i.e., by eluting with pure water). In some embodiments pertaining to biotinylated oligonucleotides, a sample may be eluted by elevating the column temperature (i.e., melting the hybridized oligonucleotide).

In some embodiments, the target analyte is eluted from the column using a step elution, wherein the mobile phase is switched from the binding buffer to the elution buffer. In some embodiments, the target analyte is eluted from the column using a gradient elution, wherein the binding buffer is transitioned to the elution buffer as a gradient.

In one aspect, the affinity chromatographic columns of the present technology are able to be re-used for affinity capture assays. In this regard, after the elution of the target analyte from the column, the column is then re-equilibrated. Re-equilibration involves flowing buffer through the column such that any residual material from the previous assay is removed. In some embodiments, re-equilibration is performed with the binding buffer. In some embodiments, re-equilibration is performed with an equilibration buffer. The equilibration buffer may have a pH that is equal to or lower than the binding buffer. In one aspect, the present technology allows for rapid re-equilibration of the affinity chromatographic column. In some embodiments, the affinity chromatographic column is re-equilibrated for 1-10 minutes, 1-2, 2-3, 3-4, 4-5, 5-6, 6-7, 7-8, 8-9, or 9-10 minutes. In some embodiments, it may be required that the chromatographic column is re-equilibrated for longer periods of time.

Following re-equilibration, the affinity chromatographic column is ready to be re-used for affinity capture assays. Affinity chromatographic columns of the instant technology can be re-used 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, or 200 or more times without degradation or noticeable loss of specificity. In one aspect, the affinity chromatographic column of the instant technology can be stored in-between uses. Buffers suitable for the storage of affinity chromatographic columns are well known in the art.

### METHODS AND EXAMPLES

### Example 1: Addition of an Epoxy Linker to Hydrophilic Particles

The nonporous, epoxy-modified hydrophilic particles for use in the disclosed methods were prepared as follows. As a first step, 1500 g of reagent alcohol (90% ethanol, ˜5% methanol, and ˜5% isopropanol), 45.1 g of polyvinylpyrrolidone (PVP-40), 4.8 g of 2,2′-Azobis(2-methylpropionitrile), 5.9 g of Triton™ N-57, and 81.7 g of styrene were charged into a reactor. After the reactor was purged with nitrogen gas, the reaction mixture was heated to and maintained at 70° C. with stirring for 3 hours.

After three hours, a solution containing 110.4 g of divinylbenzene 80 (DVB), 39.7 g of PVP-40, 510 g of reagent alcohol, and 100.2 g of p-xylene was added to the reaction mixture at a constant flow rate over two hours. Following this step, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours.

The reaction mixture was maintained at 70° C. for a total of 20 hours, after which the particles were separated from the reaction slurry by filtration. The particles were then washed sequentially with methanol, tetrahydrofuran (THF), and acetone. The final product was dried in a vacuum oven at 45° C., resulting in monodisperse 3.5 μm polymer particles. These particles contain a gradient polystyrene/DVB core with a poly(GMA/EDMA) primer (FIG. 1A). While the above reaction conditions generate 3.5 μm polymer particles, it is understood that particles ranging in sizes from 1.0 μm to 10 μm (e.g., 1.5 μm to 8 μm, such as 3.5 μm) are within the scope of the disclosure. By altering the concentrations of PVP-40, 2′2-Azobis (2-methylpropionitrile), and Triton N-57, one of ordinary skill in the art could generate a range of particle sizes.

The resultant 3.5 μm, polystyrene/DVB particles with the poly(GMA/EDMA) primer were then coated with a hydrophilic layer. 70 g of the particles were hydrolyzed in 0.5M H₂SO₄ at 60° C. for 1-20 hours. The hydrolyzed particles were washed sequentially with MilliQ water and methanol, and then dried under vacuum at 45° C. overnight. The dried particles were added into a 1 L three-necked round bottom flask with an overhead stirring motor, stirring shaft, and stir blade, a water cooled condenser, a nitrogen inlet, and a probe-controlled heating mantle. 700 mL of anhydrous diglyme (diethylene glycol dimethyl ether) was added, the flask sealed, and purged with nitrogen for 15 minutes with moderate stirring. 2.0 g of potassium tert-butoxide was added, and the reaction was raised to 70° C. To generate the hydrophilic layer, a mixture of 10.5 g glycidol, 2.6 g of glyceroltriglycidyl ether, and 14.9 g of anhydrous diglyme was prepared separately and added to the particle mixture in four equal aliquots in 30 minute intervals. The reaction was held at 70° C. for 20 hours, cooled to RT, and filtered. The resulting particles were washed sequentially with water 6 times, methanol 3 times, and then dried under vacuum overnight at 45° C. The following procedure results in a hydrophilic layer that is 2-4% (by weight) of the entire particle (FIG. 1A).

20 g of the resultant 3.5 μm particles with the hydrophilic coating were added to a mixture of 100 g of ethylene glycol diglycidyl ether (EGDGE) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable functionalization of the particle surface (FIG. 1A).

Alternatively, 20 g of the resultant 3.5 μm particles with the hydrophilic coating were added to a mixture of 100 g of poly(ethylene glycol) diglycidyl ether (a compound of Formula 1, wherein n is 4, also known as PEGDE 200) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable 3.3 ug streptavidin coverage per mg particle.

Alternatively, 20 g of the resultant 3.5 μm particles with the hydrophilic coating were added to a mixture of 100 g of poly(ethylene glycol) diglycidyl ether (a compound of Formula 1, wherein n is 9, also known as PEGDE 400) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable 3.4 ug streptavidin coverage per mg particle.

### Example 2: Preparation of a Streptavidin Column

Particles were prepared as described in Example 1 and functionalized with streptavidin. 1.5 g of particles were mixed in 7 mL of a 50-100 mM buffer (pH 8-9.2). To this, 1.5 mL of a 10 mg/mL solution of streptavidin (15 mg) was added. Next, 21.4 mL of a buffer containing a salting out agent was added dropwise. The reaction was then stirred for 20 hours between 24-37° C. The buffer system, salting out agent and its concentration together I temperature of the reaction can be adjusted to manipulate the extent of streptavidin coverage on a given particle, as shown in Table 1.

Following the 20 h incubation, 1 g of ethanolamine in 4 mL of a buffer solution (e.g., sodium phosphate) was added and the reaction stirred at RT for 3 hours. Particles were then isolated by filtration and washed. The washing process comprises: step 1: three times pH 4 water (i.e., adjusted with HCl); step 2: three times with water or water/organic solvent mixture as described in Table 1; step 3: three times with water; and step 4: twice with storage buffer (100 mM PBS, PH 7.3, 0.02% sodium azide). The particles were stored in a sealed container as a slurry in storage buffer (˜10 mL buffer/g of particle) at 4° C. Streptavidin coverage of the particles was determined using a standard bicinchoninic acid assay (BCA). Maximum binding capacity was estimated using the ratio of the molecular weight of streptavidin and a biotinylated antibody multiplied by the binding valency of streptavidin (4) as shown in Table 1.

TABLE 1
Particles Functionalized with Streptavidin

| | | | | | Streptavidin | Maximum Binding |
|---|---|---|---|---|---|---|
| | | | | Particle | Coverage | Capacity |
| Product | Buffer | Salting | | Wash 2^{nd} | (μg/mg | (μg IgG/mg |
| # | System | Out Agent | Temp | Step | particle) | particle) |
| 2a | Phosphate | 1.7M | 24° C. | Water | 2.2 | 24 |
| | Buffer | Ammonium | | | | |
| | (pH 8) | Sulfate | | | | |
| 2b | Phosphate | 2.85M | 24° C. | Water | 3.3 | 36 |
| | Buffer | Ammonium | | | | |
| | (pH 8) | Sulfate | | | | |
| 2c | Phosphate | 2.85M | 37° C. | Water | 4.6 | 50 |
| | Buffer | Ammonium | | | | |
| | (pH 8) | Sulfate | | | | |
| 2d | Phosphate | 2.85M | 37° C. | Acetonitrile/ | 5.2 | 57 |
| | Buffer | Ammonium | | Water (1/3) | | |
| | (pH 8) | Sulfate | | | | |
| 2e | Phosphate | 2.85M | 37° C. | Dimethyl | 4.6 | 50 |
| | Buffer | Ammonium | | sulfoxide/ | | |
| | (pH 8) | Sulfate | | Water (1/9) | | |
| 2f | Carbonate- | 1.5M | 37° C. | Water | 3.9 | 43 |
| | bicarbonate | Sodium | | | | |
| | Buffer | Sulfate | | | | |
| | (pH 9.2) | | | | | |
| 2g | Carbonate- | 1.5M | 37° C. | Dimethyl | 4.0 | 44 |
| | bicarbonate | Sodium | | sulfoxide/ | | |
| | Buffer | Sulfate | | Water (1/9) | | |
| | (pH 9.2) | | | | | |
| 2h | Carbonate- | 1.5M | 24° C. | Dimethyl | 3.4 | 37 |
| | bicarbonate | Sodium | | sulfoxide/ | | |
| | Buffer | Sulfate | | Water (1/9) | | |
| | (pH 9.2) | | | | | |

### Example 3: Preparation of an anti-Insulin Affinity Chromatographic Column

Particles prepared as described in Example 2 using 2.85M ammonium sulfate at 24° C. (product #2b) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and purged with 100 mM sodium phosphate buffer (pH 7.4) at 0.1 mL/min. Next, a 5 μL injection of a 0.5 mg/mL solution of biotinylated anti-insulin antibody was injected onto the column and flowed for 0.1 mL/min for 2 mins. These injections were repeated and the effluent was monitored across 140 injections using a UV detector (280 nm). The UV detector allows for the measurement of the percentage of antibody eluted.

It was shown that the biotinylated, anti-insulin antibody was binding to the streptavidin-functionalized beads of the column as indicated by the low level of antibody eluting from the column during the initial injections. After ˜90 injections, the amount of biotinylated antibody in the effluent increased, indicating that excess biotinylated antibody was not binding to the column and that the streptavidin sites were saturated (FIG. 2). Based on these results, it was estimated that ˜300 μg of the biotinylated antibody was bound to the column device. The column was washed with phosphate buffer and then storage buffer.

### Example 4: Insulin Binding on Anti-Insulin Affinity Chromatographic Column

The column of Example 3 was used to perform affinity capture of insulin. The column was connected to a liquid chromatography instrument and equilibrated with a 100 mM sodium phosphate buffer mobile phase (pH 7.4) for 2 minutes at a 0.02 mL/min flow rate.

Once equilibrated, a 5 μL injection of 0.025 μg/mL insulin was injected onto the column and flowed for 4 minutes at a 1 mL/min flow rate to allow for binding. The column effluent was monitored via a fluorescent detector (excitation 280 nm, emission 350 nm). After the 4 minute binding phase, the elution buffer (20 mM Sodium Phosphate, 500 mM NaCl; pH 2.3) was flowed onto the column at a 1 mL/min flow rate for 6 minutes. Following this, the column was re-equilibrated with 100 mM sodium phosphate buffer (pH 7.4) for 6-8 minutes at a 1 mL/min flow rate.

After re-equilibrating the column, a 5 μL injection of 0.1 μg/mL insulin was injected onto the column, and the binding, elution, and equilibration processes were repeated as described above. This was repeated using insulin concentrations of 0.25, 0.5, 0.75, 1, 1.5, and 2 μg/mL insulin.

The column of Example 3 provided for robust insulin quantification at low volumes and low concentrations. The insulin eluted from the column in less than 1 minute with sharp, well-defined peaks (FIG. 3A). These data further enabled robust titer analysis, with an R² of 0.9948 (FIG. 3B).

### Example 5: Preparation of an Anti-AAV Affinity Chromatographic Column

A column of particles functionalized with streptavidin was prepared as described in Example 2 (i.e., product #2b). The column was then equilibrated in 0.1M phosphate buffer (pH 7.5) on a liquid chromatography device at 25° C. with a flow rate of 0.1 mL/min. A 1.5 μL injection of a 1 mg/mL solution of biotinylated anti-AAV monobody was injected onto the column. This process was repeated with 100 injections, representing a 150 μg total mass load. Effluent was monitored using a UV detector (280 nm). As a control, 5 injections were analyzed by the UV detector via PEEK union, bypassing the column itself, representing 100% expected signal for unretained AAVX (FIG. 4A).

It was shown that biotinylated anti-AAV monobody was binding to the streptavidin-functionalized beads of the column as indicated by the low level of antibody eluting from the column during the initial injections. After ˜70 injections (105 μg of anti-AAV antibody), the amount of biotinylated antibody in the effluent increased, indicating that excess biotinylated antibody was not binding to the column and that the streptavidin sites were saturated (FIG. 4A).

To increase the amount of antibody bound to the functionalized particles, a column packed with particles having a greater surface coverage of streptavidin was investigated. That is, a column was packed with particles prepared as described in Example 2, product #2c, which has about twice the maximum binding capacity as product #2b. The solution of biotinylated anti-AAV monobody was injected using the same process as was used for the column containing product #2b particles as shown in FIG. 4B. The column packed with particles of product #2c did not show breakthrough until the experiment was re-started after 100 injections as demonstrated in FIG. 4C (which provides a chromatogram of additional injections following the first 100 injections from FIG. 4B). This column was loaded with AAVX nanobody after approximately 140 injections, providing about 2× amount of immobilized AAVX on the stationary phase of the column packed with product #2c versus the column packed with product #2b.

### Example 6: AAV Binding on Anti-AAV Affinity Chromatographic Column

The column of Example 5 was used to perform affinity capture of AAV9 capsids. A solution of AAV9 capsids in binding buffer (0.1M phosphate, pH 7.5) was injected onto the column at a 0.2 mL/min flow rate at 25° C. Both a step elution and gradient elution approach were tested.

For the step elution, the mobile phase was changed to the elution buffer (1% phosphoric acid) and flowed for 5 minutes at a 0.2 mL/min flow rate at 25° C. following injection of the sample. Following the 5 minute elution, the mobile phase was switched back to binding buffer and washed for 10 minutes (˜67 column volumes) prior to the next injection.

For the gradient elution, a gradient of the binding and elution buffer was flowed over the column for 2 minutes, followed by 3 minutes of elution buffer. Following the 5 minute elution, the mobile phase was switched back to binding buffer and washed for 10 minutes (˜67 column volumes) prior to the next injection.

Both the step and gradient elution methods were effective at eluting the AAV9 capsids. Notably, a low pH buffer was required for elution of the capsids from the column, as 60 mM acetic acid (pH 2.9) and 0.1M phosphate buffer (pH 3) did not result in any elution. Without being bound by theory, it is presumed this is due to a strong affinity between the capsids and the monobody used.

While the gradient elution method shifted the elution time slightly, it provided a superior baseline level for integration (FIG. 5A). To generate a titration curve, 0.5, 1, 3, 5, 8, and 10 μL of AAV9 capsid were loaded onto the column, using the bind, elute, and equilibrate processes as described above and in Example 4. This experiment was performed using both ascending, low-to-high concentrations (0.5 to 10 μL) or descending, high-to-low concentrations (10 to 0.5 μL). The descending, high-to-low approach produces a more linear titration curve, enabling titration analysis of AAV9 capsids at a range of concentrations (FIG. 5B).

### Example 7: Comparison of Affinity Chromatographic Columns to Existing AAV 50 μm Particle Columns (POROS™)

To better understand the performance of the columns of the instant disclosure, a similar study as to Example 6 was performed using the 50 μm POROS™ GoPure™ AAVX pre-packed columns (0.5×5 cm plastic hardware). This column uses the same anti-AAV monobody as used in Example 6. Using the same liquid chromatography system as Example 6 resulted in very broad peak shapes (>1 minute) and required several hours of equilibration in binding buffer before the next injection could be performed. In comparison, columns used in Examples 4 and 6 have re-equilibration times between 6 and 10 minutes.

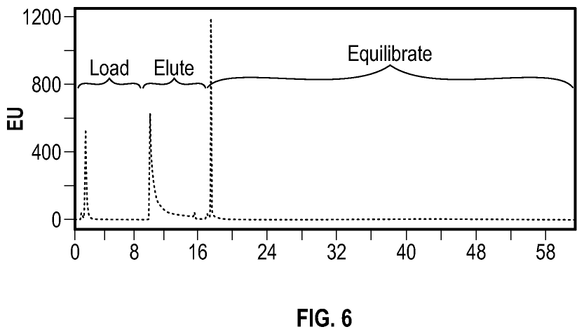

The 50 μm particles were then packed into 2.1×20 mm hardware used in Examples 2-6 and re-tested. In this experiment, the binding buffer was 10 mM Tris-HCl (pH 8.0) or 20 mM Ammonium acetate using a flow rate of 0.1-0.17 mL/min. The elution buffer was 40 mM or 60 mM acetic acid using a 0.17-0.2 mL/min flow rate. The column was re-equilibrated in the binding solution at 0.17-0.2 mL/min. Despite these modifications, broad and tailing elution peaks were still observed, and equilibration times of 40 minutes were still required (FIG. 6).

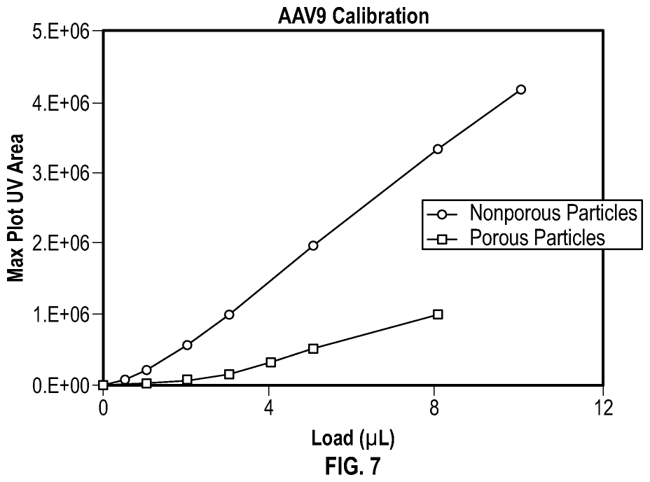

In comparing the 50 μm POROS™ beads to the nonporous polymer particles of the instant disclosure, the nonporous particles produced 3.5× more peak area with sharp, narrow peaks. In contrast, the 50 μm POROS™ beads had significant peak tailing and reduced peak area, which are not suitable for accurate integration and quantification (FIG. 7).

### Example 8: Preparation of Affinity Chromatographic Columns

Using the particles from Example 1 and the methods described in Examples 3 and 5, affinity chromatography columns are prepared using biotinylated antibodies for Tacrolimus, Troponin, cytokines, and perfluoroalkyl substances (PFAS).

### Example 9: Preparation of an Anti-Poly(A) Affinity Chromatographic Column

A column of particles functionalized with streptavidin was prepared as described in Examples 1 and 2. The column was then equilibrated in 0.1M phosphate buffer (pH 7.5) on a liquid chromatography device at 25° C. with a flow rate of 0.1 mL/min. A 2 μL injection of a 0.114 nmol/μL solution of a 5′-biotinylated dT₂₅ oligonucleotide was flowed onto the column. This process was repeated for a total of 100 injections. Effluent was monitored using a UV detector (260 nm) and the signal normalized to 5 control injections via PEEK union, bypassing the column itself, representing 100% expected signal for unretained dT₂₅ oligonucleotide (FIG. 8). The column reached saturation after approximately 50 injections (indicated by arrow), which corresponds to approximately 11.4 nmol of dT₂₅.

### Example 10: Binding of Poly(A) Oligonucleotides to an Anti-Poly(A) Affinity Chromatographic Column

The column of Example 9 was used to perform affinity capture of poly(A) oligonucleotides (i.e., oligonucleotides which hybridize to the dT₂₅ oligonucleotide). The column of Example 9 was equilibrated in 0.1M phosphate buffer (pH 7.5) on a liquid chromatography device with a flow rate of 0.2 mL/min at 35° C. A sample comprising a poly-adenosine oligo comprising 10 adenosine nucleotides (dA₁₀) was loaded onto the column and effluent monitored with a UV detector (260 nm). After 2 minutes, a 3 minute step gradient elution method was performed, using water as an elution buffer. After the 3 minute step gradient, the column was re-equilibrated in 0.1M phosphate buffer (pH 7.5) and the process was repeated with dA₁₅, dA₂₀, and dA₃₀ samples.

FIG. 9A demonstrates that the column of the present technology effectively captures poly(A) oligonucleotides with lengths >15 nucleotides. The shorter poly(A) oligonucleotide tested (dA₁₀) was less well retained on the column. Without wishing to be bound by theory it is believed that hybridization is less stable for oligos having a length less than dA₁₅. However, using the columns and methods of the present technology shorter length oligos (e.g., dA₁₀ and dA₂₋₈) can be resolved in comparison to dA₁₅ and dA₂₀ species (see, FIG. 9A and FIG. 9B).

The column of Example 9 was further used to perform affinity capture of Erythropoietin (EPO) mRNA, which comprises a poly(A) tail. The column was equilibrated as described above. A sample comprising 0.05 μg of EPO mRNA was loaded onto the column and effluent monitored with UV (260 nm). After 2 minutes, a 3 minute step gradient elution method was performed, using water as an elution buffer. After the 3 minute step gradient, the column was re-equilibrated in 0.1M phosphate buffer (pH 7.5) and the process was repeated with 0.5 μg and 2.5 μg of EPO mRNA. As shown in FIG. 10A, the column of the instant technology produced two peaks using this method, with peak 2 representing full-length EPO mRNA. Peak 1, which elutes prior to the elution gradient, represents EPO mRNA impurities (such as EPO mRNA lacking the poly(A) tail. The relative ratio of peak 1 and peak 2 is consistent with increasing mass load, suggesting that peak 1 is not breakthrough of target mRNA. Further, as shown in FIG. 10B, UV spectra of both peak 1 and peak 2 indicate predominantly nucleic acid species being present. That is, peak 1 corresponds to a “tail-less” version of mRNA or fragmented mRNA that has lost its poly(A) tail.

The column of Example 9 was further used to perform affinity capture of various mRNAs, including EPO mRNA, Firefly Luciferase (FLuc) mRNA, Cas9 mRNA, and Firefly Luciferase beta (FLucBeta) mRNA. For each sample, 2.5 μg of mRNA was used. The column and samples were prepared as described above. As shown in FIG. 11, the column of the instant technology efficiently captured each of the mRNAs tested: EPO (89.5% purity), FLuc (91.0% purity), Cas9 (70.3% purity), and FLucBeta (78.4% purity). Similar to the EPO mRNA, a first peak (believed to be impurities without affinity to the dT₂₅ oligonucleotide) was observed to varying extents for each of the samples tested.

To confirm the first observed peak is the result of tail-less mRNA (i.e., lacking a poly(A) tail), mRNA samples with or without poly(A) tails were assessed. A tail-less mRNA sample of 2180 nt in length (KH20, provided by Kactus Biosystems) or 546 nt in length (512B Kactus Biosystems) was assessed. The EPO mRNA (with a poly(A) tail) was used as a control. As shown in FIG. 12, the samples without a poly(A) tail were not retained on the column, and exhibited the same retention time as peak 1 of the EPO mRNA sample.

To further assess the specificity of the dT₂₅ column, EPO mRNA was used with varying amounts of dT₁₅₀ oligonucleotide. The dT₁₅₀ oligonucleotide will hybridize to the poly(A) tail of the EPO mRNA, thus inhibiting its ability to bind to the dT₂₅ column. EPO mRNA was spiked with either sub-equimolar, equimolar, or a molar excess of dT₁₅₀ oligonucleotide. The sample was heated to 60° C. and cooled to 15° C. over 3 minutes. Volume additions of dT₁₅₀ between 0.25-0.75 μL represent sub-equimolar amounts, volume additions of 1 uL dT₁₅₀ represent an equimolar amount, and volume additions of 2-4 uL dT₁₅₀ represent a molar excess amount. The samples were analyzed using size exclusion chromatography (to confirm hybridization of the dT₁₅₀ oligonucleotide to the EPO mRNA) and affinity chromatography with the column of the instant technology to assess binding capacity. The affinity chromatography was performed as described above. For the SEC analysis, a Premier Protein 250Å, 4.6×150 mm column with 1.7 μm particles was used with a 0.1M phosphate buffer (pH 7.5) mobile phase at 35° C.

FIG. 13A shows the SEC results for the various concentrations of dT₁₅₀. As expected, increased concentrations of dT₁₅₀ shifted the elution time of the EPO mRNA. When present in equimolar or excess molar concentrations, dT₁₅₀ completely inhibited the ability for the EPO mRNA to bind to the dT₂₅ column (FIG. 13B). These data confirm that the dT₂₅ column is specific to poly(A) oligonucleotide sequences.

### Example 11: Streptavidin Leaching Evaluation Method I

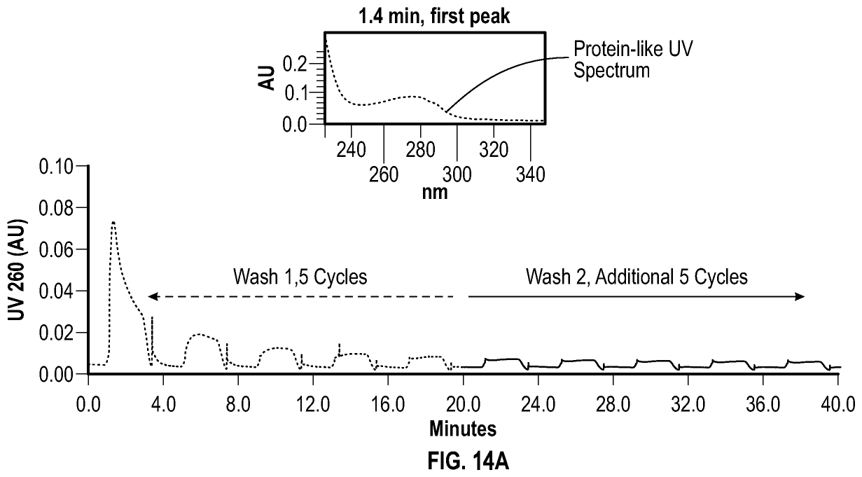

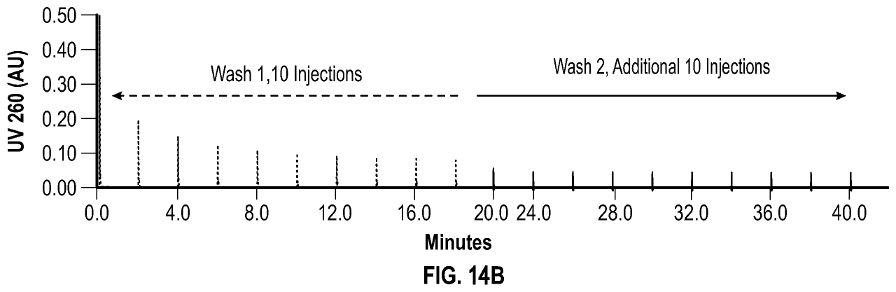

Particles (Example 2, product #2c) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH7.4) at 0.4 mL/min. Next, step gradient to 20% acetonitrile with 1% phosphoric acid was applied at 0 min and held till 2 min to wash the column. At 2.01 min the mobile phase switched back to 100 mM sodium phosphate buffer and held till 4 min. This wash cycle was repeated five times (Wash 1 labeled in FIG. 14A). Second wash was applied if needed (Wash 2, dotted line in FIG. 14A). The effluent was monitored using a UV detector (280 nm) (FIG. 14A). The inset UV spectrum in FIG. 14A shows that the leachable peak has typical UV spectra of a protein. The applied washing cycle showed a reduction in leachate.

### Example 12: Streptavidin Leaching Evaluation Method II

Particles (Example 2, product #2c) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH 7.4) at 0.4 mL/min. Next, 10 μL injection of 1% phosphoric acid aqueous solution with 20% acetonitrile was injected onto the column. These injections act as short column wash and dislodge non-covalently adsorbed leachate (streptavidin) as a sharp peak. Ten injections were executed (Wash 1, FIG. 14B). The column was then washed with 5 cycles of 1% phosphoric acid aqueous solution with 20% acetonitrile (Method I, described above), and the additional 10 injections of wash solution was repeated (Wash 2, dotted line in FIG. 14B). The effluent was monitored using a UV detector (280 nm). FIG. 14B shows the streptavidin leaching from the column of the particles of Example 2, product #2c. The Method I is an efficient approach to remove the residual (non-covalently bonded) leachates from the sorbent.

Particles (Example 2, products #2c-2h) were packed in a 2.1×20mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH 7.4) at 0.4 mL/min. Next, 10 μL injection of 1% phosphoric acid aqueous solution with 20% acetonitrile was injected onto the column and the height of the first (highest) peak was measured at 280 nm (Table 2). The data showed that the change in Step 2 particle washing step and immobilization conditions decreased the height of the first peak of the leaching. It is apparent from FIGS. 14A and 14B that the additional washes performed after sorbent packing can further suppress the leachates to approximately 10-fold lower level.

TABLE 2
The peak height measurements of the columns tested
for streptavidin leaching by Method II

| Product # | 280 nm Max |
|---|---|
| 2c | 0.52 |
| 2d | 0.22 |
| 2e | 0.04 |
| 2f | 0.32 |
| 2g | 0.32 |
| 2h | 0.36 |

### Example 13: Preparation of an Anti-Double Stranded RNA (Anti-dsRNA) Affinity Chromatographic Column

A column of particles functionalized with streptavidin was prepared as described in Example 2. The column was then equilibrated in 100 mM sodium phosphate (pH 7.4) on a liquid chromatography device with a flow rate of 0.1 mL/min. 10 μL injections of 0.73 μg/μL of anti-dsRNA antibody J2 (available from Biotechne) was flowed onto the column. This process was repeated for a total of 50 injections. Effluent was monitored using a UV detector (280 nm). As shown in FIG. 15A, the column reached saturation after approximately 40 injections, or about ˜231 ug of biotinylated anti-dsRNA antibody. No leaching was observed after coupling.

### Example 14: Binding of dsRNA to an Anti-Double Stranded RNA (Anti-dsRNA) Affinity Chromatographic Column

The column of Example 13 was used to perform affinity capture of a double-stranded RNA (dsRNA) 20KH. The column was equilibrated with 100 mM sodium phosphate (pH 7.4) on a liquid chromatography device with a flow rate of 0.2 ml/min. Different amounts of the dsRNA were loaded and eluted from the column, including 0.01 ug, 0.02 ug, 0.025 ug, 0.05 ug, 0.1 ug, 0.2 ug, 0.25 ug, and 0.5 ug. Samples were eluted using a single injection of 50 uL of water. Eluent was monitored using a UV detector (260 nm). FIG. 15B demonstrates that the affinity chromatographic column can efficiently bind and elute the dsRNA sample at a range of concentrations with sharp peaks and high resolution. There was a strong linear correlation between peak area and the mass of the dsRNA as shown in FIG. 15C.

### Example 15: Biotin Endcapping of Affinity Chromatographic Columns

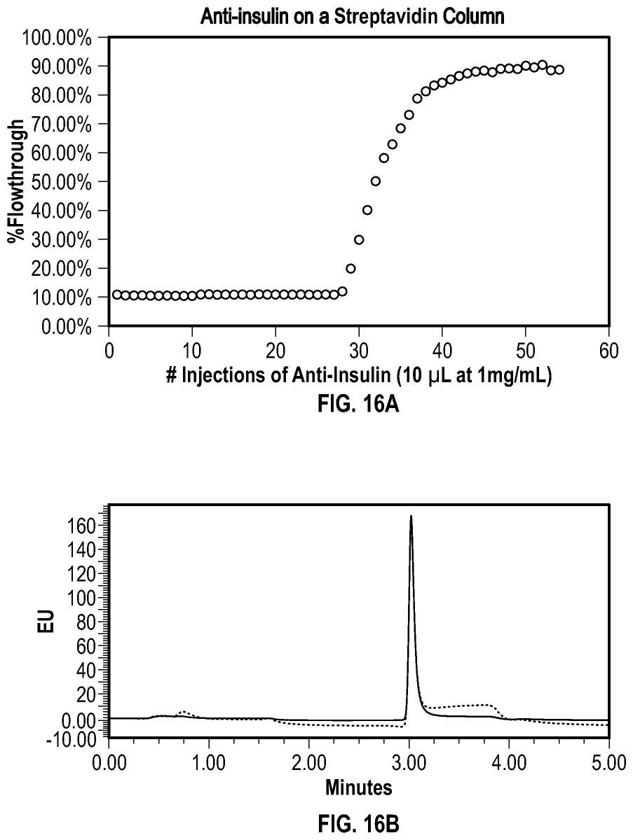

A column of particles functionalized with streptavidin was prepared as described in Example 2. The column was then equilibrated in 100 mM sodium phosphate (pH 7.4) on a liquid chromatography device with a flow rate of 0.1 mL/min. 10 μL injections of 1 mg/mL of anti-insulin antibody was flowed onto the column. This process was repeated for a total of ˜55 injections. Effluent was monitored using a UV detector (280 nm). As shown in FIG. 16A, the column reached saturation after approximately 45 injections, or about ˜400 μg of biotinylated anti-insulin antibody. No leaching was observed after coupling.

The affinity chromatographic column was next used to test the impact of biotin endcapping. 10 uL of a 1 mg/mL insulin sample were injected onto the column using 100 mM Sodium Phosphate (pH 4) at a flow rate of 0.1 mL/min for minutes 0-2 and at a flow rate of 1 mL/min for minutes 2-3. The eluent was monitored using a fluorescence detector at 280 nm excitation and 350 nm emission. The insulin was eluted from the column using 20 mM sodium phosphate (pH 2.3) 500 mM NaCl for minutes 3-4 at 1 mL/min, resulting in the chromatograph represented by the dashed line in FIG. 16B. The column was then equilibrated with 100 mM sodium phosphate (pH 4) for minutes 4-5, after which 4 injections of 1.25 ug biotin were performed (i.e., the affinity chromatograph was endcapped with biotin, wherein the biotin can bind to unoccupied streptavidin binding sites in the column). Said unoccupied streptavidin binding sites may be present due to, for example, incomplete saturation of the column or due to steric hindrance with the affinity group. The insulin bind and elute method was repeated, resulting in the chromatograph represented by the solid line in FIG. 16B. The endcapped column resulted in a flatter baseline, which can allow for better quantification, particularly for lower abundance samples. As the endcapping process is independent of affinity group, the biotin endcapping process can be used with any of the affinity chromatographic columns described herein, including but not limited to those described in Examples 3-6, 8, 9-10, and 16-17.

### Example 16: Preparation of an Anti-HCP Affinity Chromatographic Column

A column of particles functionalized with streptavidin was prepared as described in Example 2. The column was then equilibrated in 1× PBS (pH 7.4). 5 uL injections of 1 ug/uL of biotinylated anti-HCP antibodies were flowed onto the column. This process was repeated for ˜120 injections using a flow rate of 0.1 mL/min. Effluent was monitored using a UV detector (280 nm). As shown in FIG. 17A, the column reached saturation between 100-120 injections, or about 300-400 ug of biotinylated anti-HCP antibodies. The diamonds indicate the start of a new vial of anti-HCP antibodies.

### Example 17: Binding of HCPs to an Anti-HCP Affinity Chromatographic Column

The column of Example 16 was used to perform affinity capture of HCPs. The column was equilibrated with 50 mM sodium phosphate 150 mM NaCl (pH 7.4) on a liquid chromatography device for 2 minutes at 1 mL/min. Different amounts of HCP standard were loaded and eluting from the column. 5 uL of sample was injected using 50 mM sodium phosphate 150 mM NaCl (pH 7.4) at a flow rate of 0.1 mL/min. After 3 minutes, the column was washed at 1 mL/min for 2 minutes using 50 mM sodium phosphate 150 mM NaCl (pH 7.4). The HCPs were eluted from the column using 50 mM sodium phosphate 150 mM NaCl (pH 1.6) for 2 minutes at 1 mL/min. FIG. 17B shows a chromatogram for the eluted HCPs. There was a strong linear correlation between peak area and the mass of the HCPs as shown in FIG. 17C.

### OTHER EMBODIMENTS

While the technology has been described in connection with specific embodiments thereof, it will be understood that it is capable of further modifications and this application is intended to cover any variations, uses, or adaptations of the technology following, in general, the principles of the technology and including such departures from the technology that come within known or customary practice within the art to which the technology pertains and may be applied to the essential features hereinbefore set forth, and follows in the scope of the claims.

Other embodiments are within the claims.

1-43. (canceled)

44. An affinity chromatographic column comprising a column body formed of a metal or a metal alloy, the column body housing a plurality of streptavidin-conjugated nonporous particles, wherein at least 50% of accessible binding sites of the streptavidin molecules are bound with a biotinylated affinity agent or free biotin.

45. The affinity chromatographic column of claim 44, wherein each particle of the plurality of streptavidin-conjugated nonporous particles comprises:
a nonporous polymer core;
a hydrophilic surface on an outer layer of the nonporous polymer core, wherein one or more streptavidin molecules are conjugated to the hydrophilic surface; and
wherein the particle has an average particle size between 1.0 μm to 10 μm.

46. The affinity chromatographic column of claim 45, wherein the nonporous polymer core has a gradient composition.

47. The affinity chromatographic column of claim 45, wherein the nonporous polymer core comprises divinylbenzene monomers and styrene monomers.

48. The affinity chromatographic column of claim 45, wherein the hydrophilic surface is selected from the group consisting of: (3-glycidyloxypropyl) trimethoxysilane, (3-glycidyloxypropyl)triethoxysilane, polyacrylate, glycidol, glyceroltriglycidyl ether, and poly(methyl acrylate).

49. The affinity chromatographic column of claims 45, wherein the one or more streptavidin molecules are conjugated to the hydrophilic surface of the particle via an epoxy linker.

50. The affinity chromatographic column of claim 49, wherein the epoxy linker has a formula of 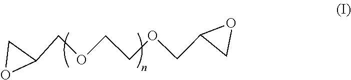
wherein n is between 1-12.

51. The affinity chromatographic column of claim 45, wherein the plurality of streptavidin molecules conjugated to the hydrophilic surface provides a surface coverage of from about 2 μg/mg particle to about 6 μg/mg particle.

52. The affinity chromatographic column of claim 44, wherein the column is characterized by a reduction in detectable leachate of streptavidin as determined by UV absorbance at about 280 nm.

53. The affinity chromatographic column of claim 52, wherein the column is characterized by a UV absorbance of leachate value of <10 mAU.

54. The affinity chromatographic column of claim 52, wherein the column is characterized by at least an 85% reduction in UV absorbance of the leachate as compared to a column that does not comprise streptavidin molecules that are bound with a mixture of the biotinylated affinity agent and the free biotin.

55. The affinity chromatography column of claim 52, wherein there is no detectable UV absorbance of leachate.

56. The affinity chromatographic column of claim 44, wherein the biotinylated affinity agent is a biotinylated antibody or biotinylated antigen-binding fragment thereof, or a biotinylated oligonucleotide.

57. A method of enriching a target analyte, the method comprising:
i) providing the affinity chromatographic column of claim 44;
ii) washing the affinity chromatographic column with a wash buffer;
iii) applying a solution containing the target analyte to the affinity chromatographic column; and
iv) washing the affinity chromatographic column with an elution buffer such that the target analyte is eluted from the column.

58. The method of claim 57, further comprising monitoring the eluent during step ii) with a UV detector configured to measure absorbance at about 280 nm.

59. The method of claim 58, wherein there is a reduction in detectable leachate of streptavidin as determined by UV absorbance during step ii).

60. The method of claim 59, wherein the UV absorbance of leachate is <10 mAU during step ii).

61. The method of claim 59, wherein there is at least an 85% reduction in the UV absorbance of leachate as compared to a column that does not comprise streptavidin molecules that are bound with a mixture of the biotinylated affinity agent and the free biotin.

62. The method of claim 59, wherein leachate absorbance is determined at the 4^{th} peak eluted from the column.

63. The method of claim 59, wherein there is no detectable UV absorbance of leachate during step ii).
